(19) United States
(12) Patent Application Publication
(10) Pub. No.: US 2013/0288320 A1
(43) Pub. Date: Oct. 31, 2013

(54) METHODS AND MICROORGANISMS FOR INCREASING THE BIOLOGICAL SYNTHESIS OF DIFUNCTIONAL ALKANES

(71) Applicant: BIOAMBER INC., Plymouth, MN (US)

(72) Inventor: Man Kit Lau, Minneapolis, MN (US)

(73) Assignee: BioAmber Inc., Plymouth, MN (US)

(21) Appl. No.: 13/868,213

(22) Filed: Apr. 23, 2013

Related U.S. Application Data
(60) Provisional application No. 61/639,390, filed on Apr. 27, 2012.

(51) Int. Cl. C12P 7/44

(52) U.S. Cl. CPC C12P 7/44
USPC 435/142; 435/167; 435/252.3; 435/252.33; 435/252.31; 435/254.2; 435/325; 435/348; 435/254.21; 435/254.23; 435/254.22

(57) ABSTRACT

A method of increasing the production a difunctional alkane in a microorganism that produces a difunctional alkane from alpha-keto acid by increasing the production of homocitrate in the cell relative to a wild-type or parent cell. The production of homocitrate may be obtained by engineering pathways that increase the production of alpha-ketoacid, such as alpha-ketoglutarate.

### TECHNICAL FIELD

Aspects of this disclosure relate to methods for increasing the production of difunctional alkanes in recombinant host cells. In particular, aspects of the disclosure describe components of genes associated with the difunctional alkane production from carbohydrates feedstocks in host cells. More specifically, aspects of the disclosure describe metabolic pathways for increasing the production of adipic acid, aminocaproic acid, caprolactam, hexamethylenediamine.

### BACKGROUND

Crude oil is the number one starting material for the synthesis of key chemicals and polymers. As oil becomes increasingly scarce and expensive, biological processing of renewable raw materials in the production of chemicals using live microorganisms or their purified enzymes becomes increasingly interesting. Biological processing, in particular, fermentations have been used for centuries to make beverages. Over the last 50 years, microorganisms have been used commercially to make compounds such as antibiotics, vitamins, and amino acids. However, the use of microorganisms for making industrial chemicals has been much less widespread. It has been realized only recently that microorganisms may be able to provide an economical route to certain compounds that are difficult or costly to make by conventional chemical means.

### SUMMARY

We provide methods of increasing the production a difunctional alkane in a recombinant host cell that produces a difunctional alkane from an alpha-ketoacid wherein the host cell has a deficiency in alpha-ketoglutarate dehydrogenase (sucA) activity. Additionally, we provide methods wherein the activity of isocitrate lyase is increased.

We further provide methods for increasing the production a difunctional alkane in a recombinant host cell that produces a difunctional alkane from an alpha-ketoacid wherein the recombinant host cell further has a deficiency in the activity of one or more enzymes selected from the group consisting of pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (ldhA).

### BRIEF DESCRIPTION OF THE DRAWINGS

FIG. 1 is a schematic diagram of an exemplary biosynthetic pathway for the production of adipic acid from glucose.

FIG. 2. is a schematic diagram of plasmid pBA006 constructed to include E. coli codon-optimized homocitrate synthase (nifV) and homoisocitrate dehydrogenase (aksF_Mm) genes.

FIG. 3. is a schematic diagram of plasmid pBA008 constructed to include E. coli codon-optimized homocitrate synthase (nifV), homoisocitrate dehydrogenase (aksF_Mm), and homoaconitase (aksED_Mm) genes.

FIG. 4. is a schematic diagram of plasmid pBA019 constructed to include an E. coli codon-optimized homoaconitase (aksED_Mj) gene.

FIG. 5. is a schematic diagram of plasmid pBA029 constructed to include E. coli codon-optimized homocitrate synthase (nifV), homoisocitrate dehydrogenase (aksF_Mm), and homoaconitase (aksED_Mj) genes.

FIG. 6. is a schematic diagram of plasmid pBA021 constructed to include an E. coli codon-optimized ketoisovalerate decarboxylase gene (kivD).

FIG. 7. is a schematic diagram of plasmid pBA042 constructed to include an E. coli codon-optimized adipate semialdehyde dehydrogenase gene (chnE) gene.

FIGS. 8A and 8B show the results of an adipate semialdehyde dehydrogenase (ChnE) enzyme assay at 340 nm with either adipate semialdehyde and NAD+ (FIG. 8A) or adipate and NADH (FIG. 8B) as the substrate.

FIG. 9 is an SDS-PAGE of the insoluble and soluble fraction of cell lysates of BL21 cells transformed with either pET28a (control), pBA049, pBA050, pBA032 or pBA042 plasmid constructs.

FIG. 10 is a graph showing a calibration curve for adipic acid.

FIG. 11 is a GS/MS chromatogram comparing adipic acid production from alpha-ketoglutarate in shake flasks of BL21 cells transformed with plasmids pBA029 and pBA021 to BL21 cells transformed with an empty control plasmid.

FIG. 12 is a GS/MS chromatogram comparing adipic acid production from glucose in fermentor-controlled conditions of BL21 cells transformed with plasmids encoding pBA029 and pBA021 to BL21 cells transformed with an empty control plasmid.

FIG. 13 is a schematic diagram of metabolic pathways in an engineered microorganism.

FIG. 14 is a photograph of a series of samples of fermentation medium and shakeflask medium showing relative alpha-ketoglutarate concentration by a color indicator, with color intensity correlating to higher alpha-ketoglutarate concentration.

FIG. 15 is a schematic diagram of metabolic pathways in an engineered microorganism.

FIG. 16 is table of reactions showing conversions of substrates that are catalyzed by enzymes that may be used in the modified microorganisms of this disclosure.

FIG. 17 is a schematic diagram of plasmids pBA049 and pBA050 constructed to include either a ketoisovalerate decarboxylase gene (kivD) (pBA049) or an alpha-keto acid decarboxylase (kdcA) (pBA050).

### DETAILED DESCRIPTION

We provide methods and materials for increasing the production of organic compounds, such as, for example, alkanes, from a carbohydrate source by a microorganism that produces a difunctional alkane using alpha-ketoacid as a precursor. The alpha-ketoacid may be alpha-ketoglutarate, alpha-ketoadipate, alpha-ketopimelate, alpha-ketosuberate, and the like. In particular, we provide microorganisms engineered or modified to express enzymes in a biosynthesis pathway that produce C5 to C8 organic compounds of interest at higher yields. We also provide methods and biosynthetic pathways that produce organic compounds of interest with higher yields.

Organic compounds of interest generally include but are not limited to difunctional alkanes, diols, and dicarboxylic acids. As used herein “difunctional alkanes” refers alkanes having two functional groups. The term “functional group” refers, for example, to a group of atoms arranged in a way that determines the chemical properties of the group and the molecule to which it is attached. Examples of functional groups include halogen atoms, hydroxyl groups (—OH), carboxylic acid groups (—COOH) and amine groups (—NH2) and the like. Preferred difunctional n-alkanes have hydrocarbon chains Cₙ in which n is a number of from about 1 to about 8, such as from about 2 to about 5 or from about 3 to about 4, but preferably 6. In a preferred example, the difunctional n-alkanes are derived from an alpha-keto acid.

In some aspects, our methods incorporate modified microorganisms capable of producing one of the following difunctional alkanes of interest, particularly, adipic acid, amino caproic acid, HMD, 6-hydroxyhexanoate. Several chemical synthesis routes have been described, for example, for adipic acid and its intermediates such as muconic acid and adipate semialdehyde; for caprolactam, and its intermediates such as 6-amino caproic acid; for hexane 1,6 diamino hexane or hexanemethylenediamine; for 3-hydroxypropionic acid and its intermediates such as malonate semialdehyde, but only a few biological routes have been disclosed for some of these organic chemicals. Therefore, we provide engineered metabolic routes, isolated nucleic acids or engineered nucleic acids, polypeptides or engineered polypeptides, host cells or genetically engineered host cells, methods and materials to produce difunctional alkanes using alpha-ketoacid as a precursor from sustainable feedstock.

The term “polypeptide” and the terms “protein” and “peptide” which are used interchangeably herein, refers to a polymer of amino acids, including, for example, gene products, naturally-occurring proteins, homologs, orthologs, paralogs, fragments, and other equivalents, variants and analogs of the forgoing. Typically, a polypeptide having enzymatic activity catalyzes the formation of one or more products from one or more substrates. In some aspects, the catalytic promiscuity properties of some enzymes may be combined with protein engineering and may be exploited in novel metabolic pathways and biosynthesis applications. In some examples, existing enzymes are modified for use in organic biosynthesis. In some examples, the reaction mechanism of the enzyme may be altered to catalyze new reactions, to change, expand or improve substrate specificity. One should appreciate that if the enzyme structure (e.g. crystal structure) is known, enzymes properties may be modified by rational redesign (see US patent applications US20060160138, US20080064610 and US20080287320 the subject matter of which are incorporated by reference in their entirety).

Modification or improvement in enzyme properties may arise from introduction of modifications into a polypeptide chain that may, in effect, alter the structure-function of the enzyme and/or interaction with another molecule (e.g., substrate versus unnatural substrate). It is known that some regions of the polypeptide may enzyme activity. For example, a small perturbation in the composition of amino acids involved in catalysis and/or in substrate binding domains can have significant effects on enzyme function. Some amino acid residues may be at important positions for maintaining the secondary or tertiary structure of the enzyme, and thus also produce noticeable changes in enzyme properties when modified. In some examples, the potential pathway components are variants of any of the foregoing. Such variants may be produced by random mutagenesis or may be produced by rational design for production of an enzymatic activity having, for example, an altered substrate specificity, increased enzymatic activity, greater stability, etc. Thus, in some examples, the number of modifications to a reference parent enzyme that produces an enzyme having the desired property may comprise one or more amino acids, 2 or more amino acids, 5 or more amino acids, 10 or more amino acids, or 20 or more amino acids, up to about 10% of the total number of amino acids, up to about 20% of the total number of amino acids, up to about 30% of the total number of amino acids, up to about 40% of the total number of amino acids making up the reference enzyme or up to about 50% of the total number of amino acids making up the reference enzyme. Additionally, modifications or improvements in enzyme activity can be brought about by expression of proteins encoded by a nucleotide sequence having about 95% or more, about 90% or more, about 85% or more, about 80% or more, about 75% or more, or about 50% or more sequence identity with a nucleotide sequence encoding the reference parent enzyme.

Those skilled in the art will understand that engineered pathways exemplified herein are described in relation to, but are not limited to, species specific genes and encompass homologs or orthologs of nucleic acid or amino acid sequences. Homologous and orthologous sequences possess a relatively high degree of sequence identity/similarity when aligned using methods known in the art.

Aspects our methods and microorganisms relate to “genetically modified” or recombinant microorganisms or host cells that have been engineered to possess new metabolic capabilities or new metabolic pathways. As used herein the term “genetically modified” microorganisms includes microorganisms having at least one genetic alteration not normally found in the wild type strain of the referenced species such as expression of a recombinant gene. In some examples, genetically engineered microorganisms are engineered to express or overexpress at least one particular enzyme at critical points in a metabolic pathway, and/or suppress or block the activity of other enzymes, to overcome or circumvent metabolic bottlenecks.

The term “metabolic pathway” or “biosynthesis pathway” refers to a series of one or more enzymatic reactions in which the product of one enzymatic reaction becomes the substrate for the next enzymatic reaction. At each step of a metabolic pathway, intermediate compounds are formed and utilized as substrates for a subsequent step. These compounds may be called “metabolic intermediates.” The products of each step are also called “metabolites.” A “precursor” may be compound that serves as a substrate in a first enzymatic reaction, particularly where a product of the first enzymatic reaction is a substrate in one or more additional enzymatic reactions.

In some aspects, we provide alternative pathways for making a product of interest from one or more available and sustainable substrates in one or more host cells or microorganisms of interest. One should appreciate that an engineered pathway for making the difunctional alkanes of interest may involve multiple enzymes and therefore the flux through the pathway may not be optimum for the production of the product of interest. Consequently, in some aspects of the methods disclosed herein, flux is optimally balanced by modulating the activity level of the pathway enzymes relative to one another. In some examples, microorganisms can be modified to reduce or eliminate the activity of enzymes that act as “carbon-sinks” by diverting substrates from the desired metabolic pathway and catalyzing these substrates into compounds that can not be converted to organic compounds of interest.

We provide genetically modified host cells or microorganisms and methods of using the same to produce difunctional alkanes from alpha-ketoacid, particularly alpha-ketoglutarate. A “host cell” as used herein refers to an in vivo or in vitro eukaryotic cell, a prokaryotic cell or a cell from a multicellular organism (e.g. cell line) cultured as a unicellular entity. A host cell may be prokaryotic (e.g., bacterial such as E. coli or B. subtilis) or eukaryotic (e.g., a yeast, mammal or insect cell). For example, host cells may be bacterial cells (e.g., Escherichia coli, Bacillus subtilis, Mycobacterium spp., M. tuberculosis, or other suitable bacterial cells), Archaea (for example, Methanococcus Jannaschii or Methanococcus Maripaludis or other suitable archaic cells), yeast cells (for example, Saccharomyces species such as S. cerevisiae, S. pombe, Picchia species, Candida species such as C. albicans, or other suitable yeast species). Preferred host cells include E. coli of the BL21 strain. Eukaryotic or prokaryotic host cells can be, or have been, genetically modified (also referred as “recombinant host cell”, “metabolic engineered cells” or “genetically engineered cells”) and are used as recipients for a nucleic acid, for example, an expression vector that comprises a nucleotide sequence encoding one or more biosynthetic or engineered pathway gene products. Eukaryotic and prokaryotic host cells also denote the progeny of the original cell which has been genetically engineered by the nucleic acid. In some examples, a host cell may be selected for its metabolic properties. For example, if a selection or screen is related to a particular metabolic pathway, it may be helpful to use a host cell that has a related pathway. Such a host cell may have certain physiological adaptations that allow it to process or import or export one or more intermediates or products of the pathway. However, in other examples, a host cell that expresses no enzymes associated with a particular pathway of interest may be selected to be able to identify all of the components required for that pathway using appropriate sets of genetic elements and not relying on the host cell to provide one or more missing steps.

The metabolically engineered cell may be made by transforming a host cell with at least one nucleotide sequence encoding an enzyme involved in the engineered metabolic pathways. As used herein the term “nucleotide sequence”, “nucleic acid sequence” and “genetic construct” are used interchangeably and mean a polymer of RNA or DNA, single- or double-stranded, optionally containing synthetic, non-natural or altered nucleotide bases. A nucleotide sequence may comprise one or more segments of cDNA, genomic DNA, synthetic DNA, or RNA.

In a preferred example, the nucleotide sequence encoding enzymes or proteins in the metabolic pathway is codon-optimized to reflect the typical codon usage of the host cell without altering the polypeptide encoded by the nucleotide sequence. In selected examples, the term “codon optimization” or “codon-optimized” refers to modifying the codon content of a nucleic acid sequence without modifying the sequence of the polypeptide encoded by the nucleic acid to enhance expression in a particular host cell. In selected examples, the term is meant to encompass modifying the codon content of a nucleic acid sequence as a mean to control the level of expression of a polypeptide (e.g. either increase or decrease the level of expression). Accordingly, aspects include nucleic sequences encoding the enzymes involved in the engineered metabolic pathways. In some examples, a metabolically engineered cell may express one or more polypeptide having an enzymatic activity necessary to perform the steps described below. For example, a particular cell may comprise one, two, three, four, five or more than five nucleic acid sequences, each one encoding the polypeptide(s) necessary to perform the conversion of alpha-ketoacid into difunctional alkane. Alternatively, a single nucleic acid molecule can encode one, or more than one, polypeptide. For example, a single nucleic acid molecule can contain nucleic acid sequences that encode two, three, four or even five different polypeptides. Nucleic acid sequences useful for the methods and microorganisms described herein may be obtained from a variety of sources such as, for example, amplification of cDNA sequences, DNA libraries, de novo synthesis, and/or excision of one or more genomic segments. The sequences obtained from such sources may then be modified using standard molecular biology and/or recombinant DNA technology to produce nucleic sequences having desired modifications. Exemplary methods for modification of nucleic acid sequences include, for example, site directed mutagenesis, PCR mutagenesis, deletion, insertion, substitution, swapping portions of the sequence using restriction enzymes, optionally in combination with ligation, homologous recombination, site specific recombination or various combination thereof. In other examples, the nucleic acid sequences may be a synthetic nucleic acid sequence. Synthetic polynucleotide sequences may be produced using a variety of methods described in U.S. Pat. No. 7,323,320, the subject matter of which is incorporated herein by reference in its entirety.

Methods of transformation for bacteria, plant, and animal cells are known. Common bacterial transformation methods include electroporation and chemical modification.

We also provide expression cassettes comprising a nucleic acid or a subsequence thereof encoding a polypeptide involved in the engineered pathway. In some examples, the expression cassette can comprise the nucleic acid that is operably linked to a transcriptional element (e.g. promoter) and/or to a terminator. A promoter is a sequence of nucleotides that initiates and controls the transcription of a desired nucleic acid sequence by an RNA polymerase enzyme. In some examples, promoters may be inducible. In other examples, promoters may be constitutive. Non limiting examples of suitable promoters for the use in prokaryotic host cells include a bacteriophage T7 RNA polymerase promoter, a trp promoter, a lac operon promoter and the like. Non limiting examples of suitable strong promoter for the use in prokaryotic cells include lacUV5 promoter, T5, T7, Trc, Tac and the like. The nucleotide sequence of a suitable T5 promoter is shown in SEQ ID NO: 15. Non limiting examples of suitable promoters for use in eukaryotic cells include a CMV immediate early promoter, a SV40 early or late promoter, a HSV thymidine kinase promoter and the like. Termination control regions may also be derived from various genes native to the preferred hosts.

In some examples, a first enzyme of the engineered pathway may be under the control of a first promoter and the second enzyme of the engineered pathway may be under the control of a second promoter, wherein the first and the second promoter have different strengths. For example, the first promoter may be stronger than the second promoter or the second promoter may be stronger than the first promoter. Consequently, the level a first enzyme may be increased relative to the level of a second enzyme in the engineered pathway by increasing the number of copies of the first enzyme and/or by increasing the promoter strength to which the first enzyme is operably linked relative to the promoter strength to which the second enzyme is operably linked. In some other examples, the plurality of enzymes of the engineered pathway may be under the control of the same promoter. In other examples, altering the ribosomal binding site affects relative translation and expression of different enzymes in the pathway. Altering the ribosomal binding site can be used alone to control relative expression of enzymes in the pathway, or can be used in concert with the aforementioned promoter modifications and codon optimization that also affect gene expression levels.

In an exemplary example, expression of the potential pathway enzymes may be dependent upon the presence of a substrate on which the pathway enzyme will act in the reaction mixture. For example, expression of an enzyme that catalyzes conversion of A to B may be induced in the presence of A in the media. Expression of such pathway enzymes may be induced either by adding the compound that causes induction or by the natural build-up of the compound during the process of the biosynthetic pathway (e.g., the inducer may be an intermediate produced during the biosynthetic process to yield a desired product).

The metabolic pathways, methods, and microorganisms for the increased production of difunctional alkanes of this disclosure will now be described in detail. The methods and microorganisms disclosed herein can be advantageously used in connection with difunctional alkane-producing microorganisms that rely on alpha-keto acid chain elongation reactions. For example, alpha-ketoglutarate may serve as a precursor in at least one alpha-ketoacid elongation reaction and a product of the elongation reaction, such as alpha-ketoadipate, alpha-ketopimelate, or alpha-ketosuberate, may serve as a precursor in a reaction pathway that produces a difunctional alkane. Difunctional alkane-producing microorganisms that utilize alpha-ketoglutarate as a precursor in the production of difunctional alkanes are known in the art. Exemplary methods and microorganisms that produce a difunctional alkane from alpha-ketoglutarate are disclosed in U.S. Pat. No. 8,133,704, U.S. Pat. No. 8,192,976, and US 20110171699, which are incorporated herein by reference.

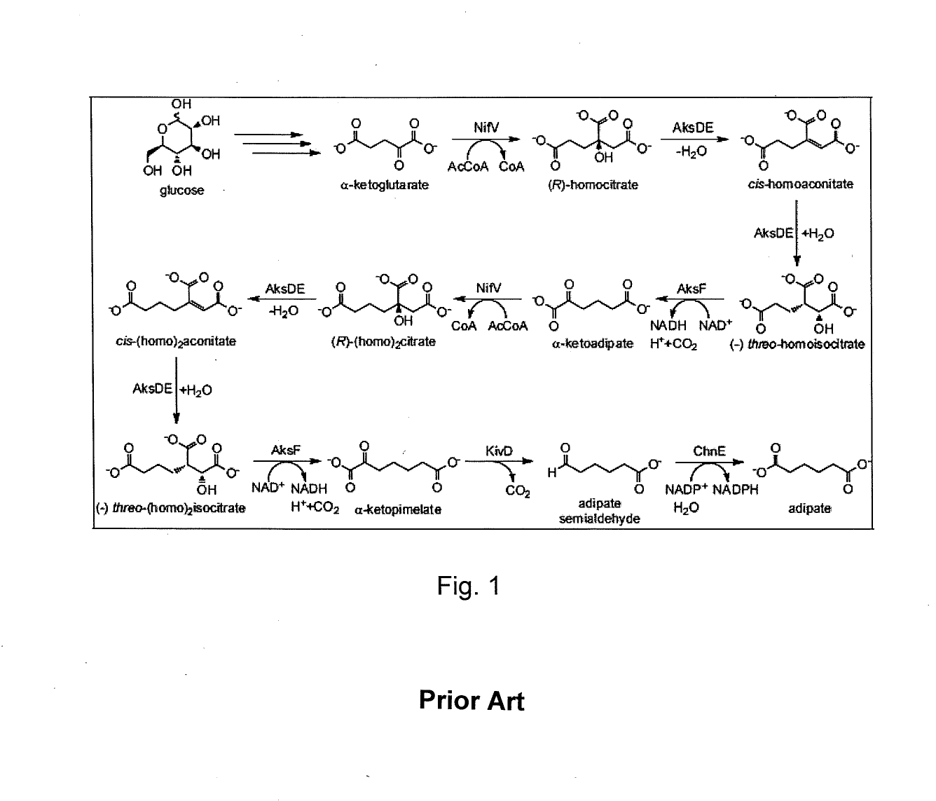

FIG. 1 shows an exemplary metabolic pathway for the biosynthesis of adipic acid using alpha-ketoglutarate as a precursor. As shown in FIG. 1, the metabolic pathway can utilize glucose as a carbon source for the production of adipic acid. Alternatively, the metabolic pathway can utilize alpha-keto acids, such as alpha-ketoglutarate or alpha-ketopimelate, as carbon sources for the production of adipic acid. In alternative examples, a combination of glucose, alpha-keto acids and/or alpha-ketopimelate may be used as carbon sources.

As shown in FIG. 1, conversion of alpha-keto acids to adipic acid requires two chain elongation reactions. Exemplary alpha-keto acid chain elongation reactions (also called 2-oxo acid elongation) are biosynthetic pathways that convert a substrate having Cₙ carbons to a product having Cₙ₊ₓ carbons, where “x” is an integer greater than or equal to 1. For example, alpha-keto acid chain elongation reactions may convert alpha-ketoglutarate (C5 chain) and acetylCoA to alpha-ketopimelate (C7 chain).

An exemplary alpha-keto acid elongation pathway comprises enzymes that catalyze the following steps:

(1) condensation of alpha-ketoglutarate and acetylCoA to form (R)-homocitrate (e.g. by action of a homocitrate synthase, such as, for example, AksA, NifV, Hcs, or Lys 20/21, preferably NifV)

(2) dehydration and hydration to (−)threo-homoisocitrate with cis homoaconitate serving as an intermediate (e.g. by action of a homoaconitase such as for example AksD/E, LysT/U, Lys4, or 3-isopropylmalate dehydratase, preferably AksD/E)

(3) oxidative decarboxylation of (−)threo-homoisocitrate to alpha-ketoadipate (e.g. by action of homoisocitrate dehydrogenase such as for example AksF, Hicdh, Lys12, 2-oxosuberate synthase, or 3-isopropylmalate dehydrogenase, preferably AksF).

(4) condensation of alpha-ketoadipate and acetylCoA to form (R)-(homo)₂citrate (e.g. by action of a homocitrate synthase, such as, for example, AksA, NifV, Hcs, or Lys 20/21, preferably NifV)

(5) dehydration and hydration to (−)threo-(homo)₂aconitate with cis-(homo)₂aconitate serving as an intermediate (e.g. by action of a homoaconitase such as for example AksD/E, LysT/U, Lys4, or 3-isopropylmalate dehydratase, preferably AksD/E)

(6) oxidative decarboxylation of (−)threo-(homo)₂aconitate to alpha-ketopimelate (e.g. by action of homoisocitrate dehydrogenase such as for example AksF, Hicdh, Lys12, 2-oxosuberate synthase, or 3-isopropylmalate dehydrogenase, preferably AksF).

Each elongation step may comprise a set of three enzymes: (1) an acyltransferase or acyltransferase homolog, (2) a homoaconitase or homoaconitase homolog, and (3) a homoisocitrate dehydrogenase or homoisocitrate dehydrogenase homolog. An enzymes that catalyzes a reaction in a first elongation reaction may be the same or different from an enzyme catalyzing the corresponding reaction in a second elongation reaction. Suitable homocitrate synthases, homoaconitases and homoisocitrate dehydrogenase are listed in Table 1, although others a possible.

TABLE 1

| Activity | Candidate enzymes |
|---|---|
| homocitrate | AksA, NifV, Hcs, Lys20/21 |
| synthase | |
| homoaconitase | AksD/E, LysT/U, Lys4, |
| | 3-isopropylmalate dehydratase Large/Small |
| homoaconitase | AksD/E, LysT/U, Lys4, |
| | 3-isopropylmalate dehydratase Large/Small |
| homoisocitrate | AksF, Hicdh, Lys 12, 2-oxosuberate synthase, |
| dehydrogenase | 3-isopropylmalate dehydrogenase |
| Homo2citrate | AksA, NifV |
| synthase | |
| Homo2aconitase | AksD/E, |
| | 3-isopropylmalate dehydratase Large/Small |
| Homo2aconitase | AksD/E, |
| | 3-isopropylmalate dehydratase Large/Small |
| Homo2isocitrate | AksF, |
| dehydrogenase | 2-oxosuberate synthase, |
| | 3-isopropylmalate dehydrogenase |

The first reaction of each elongation step is catalyzed by an acetyl transferase enzyme that converts acyl groups into alkyl groups on transfer. In some examples, the acyl transferase enzyme is a homocitrate synthase (EC 23.3.14). Homocitrate synthase enzymes catalyze the chemical reaction acetyl-CoA+H₂O+2-oxoglutarate⇄homocitrate+CoA. The product, homocitrate, is also known as (R)-2-hydroxybutane-1,2,4-tricarboxylate.

It has been shown that some homocitrate synthases, such as AksA, have a broad substrate range and catalyze the condensation of oxoadipate and oxopimelate with acetyl CoA (Howell et al., 1998, Biochemistry, Vol. 37, pp 10108-10117). Some aspects our methods provide a homocitrate synthase having substrate specificity for oxoglutarate or for oxoglutarate and for oxoadipate. Preferred homocitrate synthases are known by EC number 2.3.3.14. In general, the process for selection of suitable enzymes may involve searching enzymes among natural diversity by searching homologs from other organisms and/or creating and searching artificial diversity and selecting variants with selected enzyme specificity and activity.

For example, a homocitrate synthase askA may be derived from Methanococcus jannaschii. Methanococcus jannaschii is a thermophilic methanogen and the coenzyme B pathway in this organism has been characterized at 50-60° C. Accordingly, enzymes originating from Methanococcus jannaschii, such as homocitrate synthase askA, may have peak efficiency at higher temperatures around about 50-60° C. However, alternative AksA protein homologs from other methanogens that propagate at a lower temperature may also be used. Indeed, it is believed that recruiting alternative Aks protein homologs from other methanogens that propagate at a lower temperature might be advantageous to yield a more efficient keto-acid elongation pathway.

In some preferred examples, the first step of the elongation pathway may be engineered to be catalyzed by the homocitrate synthase NifV or NifV homologs. NifV has been shown to use oxoglutarate and oxoadipate as a substrate but has not been demonstrated to use oxopimelate as a substrate (see Zheng et al., (1997) J. Bacteriol. Vol. 179, pp 5963-5966). Consequently, an engineered 2-keto-elongation pathway comprising the homocitrate synthase NifV maximizes the availability of 2-ketopimelate intermediate.

Homologs of NifV are found in a variety of organisms including, but not limited to, Azotobacter vinelandii, Klebsiella pneumoniae, Azotobacter chroococcum, Frankia sp. (strain FaCl), Anabaena sp. (strain PCC 7120), Azospirillum brasilense, Clostridium pasteurianum, Rhodobacter sphaeroides, Rhodobacter capsulatus, Frankia alni, Carboxydothermus hydrogenoformans (strain Z-2901/DSM 6008), Anabaena sp. (strain PCC 7120), Frankia alni, Enterobacter agglomerans, Erwinia carotovora subsp. atroseptica (Pectobacterium atrosepticum), Chlorobium tepidum, Azoarcus sp. (strain BH72), Magnetospirillum gryphiswaldense, Bradyrhizobium sp. (strain ORS278), Bradyrhizobium sp. (strain BTAi1/ATCC BAA-1182), Clostridium kluyveri (strain ATCC 8527/DSM 555/NCIMB 10680), Clostridium kluyveri (strain ATCC 8527/DSM 555/NCIMB 10680), Clostridium butyricum 5521, Cupriavidus taiwanensis (strain R1/LMG 19424), Ralstonia taiwanensis (strain LMG 19424), Clostridium botulinum (strain Eklund 17B/type B), Clostridium botulinum (strain Alaska E43/type E3), Synechococcus sp. (strain JA-2-3B′a(2-13)) (Cyanobacteria bacterium Yellowstone B-Prime), Synechococcus sp. (strain JA-3-3Ab) (Cyanobacteria bacterium Yellowstone A-Prime), Geobacter sulfurreducens and Zymomonas mobilis. In preferred examples, homocitrate synthase is NifV from Azotobacter vinelandii and may have an amino acid sequence according to SEQ ID NO: 1. In other preferred examples, homocitrate synthase is NifV from Azotobacter vinelandii and is encoded by a nucleotide sequence according to SEQ ID NO: 2, which is codon-optimized for expression in E. coli.

In other examples, the first step of the pathway may be engineered to be catalyzed by the homocitrate synthase Lys 20 or Lys 21. Lys 20 and Lys 21 are two homocitrate synthase isoenzymes implicated in the first step of the lysine biosynthetic pathway in the yeast Saccharomyces cerevisiae. Homologs of Lys 20 or Lys 21 are found in a variety of organisms such as Pichia stipitis and Thermus thermophilus. Lys20 and Lys21 enzymes have been shown to use oxoglutarate as substrate, but not to use oxoadipate or oxopimelate. Consequently, engineered alpha-keto elongation pathway comprising Lys20/21 maximizes the availability of 2-oxoadipate. In some examples, enzymes catalyzing the reaction involving acetyl coenzyme A and alpha-keto acids as substrates are used to convert alpha-keto acid into homocitrate (e.g. EC 2.3.3.-). Methanogenic archaea contain three closely related homologs of AksA: 2-isopropylmalate synthase (LeuA) and citramalate (2-methylmalate) synthase (CimA) which condenses acetyl-CoA with pyruvate. This enzyme is believed to be involved in the biosynthesis of isoleucine in methanogens and possibly other species lacking threonine dehydratase. In some examples, the acyl transferase enzyme is an isopromylate synthase (e.g. LeuA, EC 2.3.3.13) or a citramalate synthase (e.g. CimA, EC 2.3.1.182).

The second step of the keto elongation pathway may be catalyzed by a homoaconitase enzyme. The homoaconitase enzyme catalyzes the hydration and dehydration reactions as shown in FIG. 1. In some examples, the homoaconitase is AksD/E, lysT/U, LysF or lys4 or homologs or variants thereof. Homoaconitases AksD/E and lysT/U have been shown to consist of two polypeptides AksD and AksE, lysT and lysU, respectively. LysT/U, LysF or lys4 are found in the lysine biosynthetic pathway of filamentous fungi and Thermus thermophipus. Lysine may be synthesized from the aminoadipate pathway and lysF (various filamentous fungi) and LysT/LysU (T. thermophilus) catalyze the formation of homoisocitrate that converts into alpha-aminoadipate for lysine synthesis (Mol Gen Genet. 1997 255 237, FEMS Microbiol. Lett. 2004, 233, 315).

In some preferred examples, the homoaconitase is AksD/E from Methanocaldococcus jannaschii and has an amino acid sequence according to SEQ ID NO: 11 (AksD) and SEQ ID NO: 12 (AksE) or Methanococcus maripaludis and has an amino acid sequence according to SEQ ID NO: 7 (AksD) and SEQ ID NO: 8 (AksE). In other preferred examples, the homoaconitase is AksD/E, preferably from Methanocaldococcus jannaschii or Methanococcus maripaludis and is encoded by the nucleotide sequences of SEQ ID NOs: 13 and 14 (Methanocaldococcus jannaschii) or SEQ ID NOs: 9 and 10 (Methanococcus maripaludis), which are codon-optimized for expression in E. coli.

The last step of each keto elongation cycle is catalyzed by a homoisocitrate dehydrogenase. A homoisocitrate dehydrogenase (e.g. EC 1.1.1.87) is an enzyme that generally catalyzes the chemical reaction:

(1R,2S)-1-hydroxybutane-1,2,4-tricarboxylate+NAD⁺ [symbol] oxoadipate+CO2+NADH+H⁺. wherein (1R,2S)-1-hydroxybutane-1,2,4-tricarboxylate is also known as (−)threo-homoisocitrate and oxoadipate is also known as alpha-ketoadipate.

In some examples, the homoisocitrate dehydrogenase may be, but is not limited to, AksF, Hicdh, lys12, LueA, LeuC, LeuD and/or LeuB (EC1.1.1.85). LeuB is 3-isopropylmalate dehydrogenase (EC1.1.1.85) (IMDH) and catalyzes the third step in the biosynthesis of leucine in bacteria and fungi, the oxidative decarboxylation of 3-isopropylmalate into 2-oxo-4-methylvalerate. It has also been shown that 2-ketoisovalerate is converted to 2-ketoisocaproate through a three step elongation cycle by LeuA (2-isopropylmalate synthase), LeuC, LeuD (3-isopropylmalate isomerase complex) and LeuB (3-isopropylmalate dehydrogenase) in the leucine biosynthesis pathway. One should appreciate that these enzymes have broad substrate specificity (see Zhang et al., (2008), P.N.A.S) and may catalyze the alpha-ketoacid elongation reactions. In some examples, LeuA, LeuC, LeuD and/or LeuB catalyze the elongation of alpha-ketoglutarate to alpha-ketoadipate and the elongation of alpha-ketoadipate to alpha-ketopimelate. Lys12 in the S. cerevisiae lysine biosynthesis catalyzes the formation of alpha-ketoadipate from homoisocitrate. HICDH from T. thermophilus is another homoisocitrate dehydrogenase in the lysine biosynthetic pathway. Unlike Lys12, HICDH has a broad substrate specificity and can catalyze the reaction with isocitrate as substrate (J. Biol. Chem. 2003, 278, 1864).

In preferred examples, the homoisocitrate dehydrogenase is AksF from Methanosarcina barkerii and has an amino acid sequence according to SEQ ID NO: 3 or from Methanococcus maripaludis and has an amino acid sequence according to SEQ ID NO: 4. In other preferred examples, the homoisocitrate dehydrogenase is AksF from Methanosarcina barkerii or Methanococcus maripaludis and is encoded by the nucleotide sequences of SEQ ID NO: 5 (Methanosarcina barkerii) or SEQ ID NO: 6 (Methanococcus maripaludis), which are codon-optimized for expression in E. coli.

Following alpha-keto chain elongation reactions, the biosynthetic pathway may include a ketopimelate decarboxylase step followed by a dehydrogenation step to convert alpha-ketopimelate to adipate with adipic semialdehyde as an intermediate.

Decarboxylation of alpha-ketopimelate may be accomplished by expressing in a host cell a protein having a biological activity substantially similar to an alpha-keto acid decarboxylase to generate a carboxylic acid semialdehyde, such as adipic semialdehyde. The term “alpha-keto acid decarboxylase” (KDCs) refers to an enzyme that catalyzes the conversion of alpha-ketoacids to carboxylic acid semialdehyde and carbon dioxide. Some KDCs of particular interest are known by the EC following numbers: EC 4.1.1.1; EC 4.1.1.80, EC 4.1.1.72, 4.1.1.71, 4.1.1.7, 4.1.1.75, 4.1.1.82, 4.1.1.74. Some KDCs have a wide substrate range whereas other KDCs are more substrate specific. KDCs are available from a number of sources, including but not limited to, S. cerevisiae and bacteria.

In some exemplary examples, suitable KDCs include but are not limited to KivD from Lactococcus lactis (UniProt Q684J7), AR0010 (UniProt Q06408) from S. cerevisiae, PDC1 (UniProt P06169), PDC5 (UniProt P16467), PDC6 (UniProt P26263), Thi3 from S. cerevisiae, kgd from M. tuberculosis (UniProt 50463), mdlc from P. putida (UniProt P20906), arul from P. aeruginosa (UniProt AAG08362), fom2 from S. wedmorensis (UniProt Q56190), Pdc from Clostridium acetobutyculum, ipdC from E. coacae (UniProt P23234) or any homologous proteins from the same or other microbial species. In some examples, the keto acid decarboxylase is a pyruvate decarboxylase known by the EC number EC 4.1.1.1. Pyruvate decarboxylases are enzymes that catalyze the decarboxylation of pyruvic acid to acetaldehyde and carbon dioxide. Pyruvate decarboxylases are available from a number of sources including but not limited to S. cerevisiae and bacteria (see US Patent 20080009609 which are incorporated herein by reference).

In some preferred examples, the alpha-keto acid decarboxylase is the alpha-ketoisovalerate decarboxylase KivD or a homolog of the KivD enzyme that naturally catalyzes the conversion of alpha-ketoisovalerate to isobutyraldehyde and carbon dioxide. In preferred examples, the ketoisovalerate decarboxylase may be KivD from Lactococcus lactis KF1247 and have an amino acid sequence according to SEQ ID NO: 16. In other preferred examples, the ketoisovalerate decarboxylase is KivD from Lactococcus lactis KF1247 and is encoded by the nucleotide sequence of SEQ ID NO: 17, which is codon-optimized for expression in E. coli.

In other examples, alpha-keto acid decarboxylase is one of the branched chain alpha-keto acid decarboxylases (EC number 4.1.1.72). For example, a branched-chain keto acid decarboxylase may be kdcA from Lactococcus lactis B 1157 and have an amino acid sequence according to SEQ ID NO: 18. In other examples, the branched-chain keto acid decarboxylase may be kdcA from Lactococcus lactis B1157 and be encoded by the nucleotide sequence of SEQ ID NO: 19, which is codon-optimized for expression in E. coli.

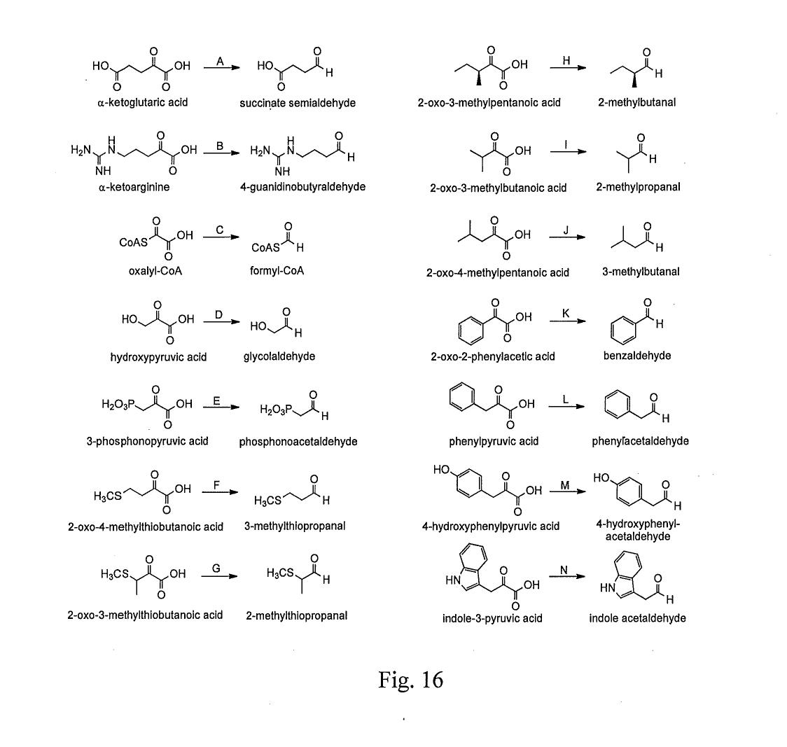

Additionally, other 2-keto-acid decarboxylases having reactivity towards alpha-ketopimelate may be used in the metabolic pathways and microorganisms of this disclosure. For example, the Kgd gene encoding alpha-ketoglutarate decarboxylase and aruI gene encoding 2-ketoarginine decarboxylase, which catalyze the conversion of alpha-ketoglutarate to succinate semialdehyde and 2-ketoarginine to 4-guanidinobutyraldehyde, respectively, may be used (FIG. 16, Reactions A and B). Alpha-ketoglutarate decarboxylase and succinate semialdehyde dehydrogenase catalyze the formation of succinic acid in Mycobacterium tuberculosis by linking the oxidative and reductive halves of the TCA cycle. In addition to M. tuberculosis Kgd, a similar decarboxylase may be derived from Bradyrhizobium japonicm, particularly strain USDA 110, the genome of which has been completely sequenced. The Kgd from B. japonicum may be codon optimized for E. coli expression. Additionally, MenD in E. coli may be another source of alpha-ketoglutarate decarboxylase enzyme and may be coupled with condensation of the thiamine-attached succinate semialdehyde with isochorismate to form an intermediate in menaquinone biosynthesis. The amino acid sequence of the MenD protein is shown in SEQ ID NO: 45. Additionally, protein engineering techniques may be employed to amend the active site for improved specificity toward alpha-ketopimelate.

Another potential enzyme for use in the metabolic pathways and microorganisms as the alpha-keto-decarboxylase is the oxalyl-CoA decarboxylase from Oxlobacter formigenes. The amino acid sequence of an exemplary oxalyl-CoA decarboxylase is shown in SEQ ID NO: 46. This enzyme catalyzes the decarboxylation of oxalyl-CoA to formyl-CoA (FIG. 16, Reaction C). The oxc gene has been cloned and expressed in E. coli and was found to form homodimers and be functionally active. Moreover, oxalyl-CoA decarboxylase may be preferred in some instances because of the sheer size of the functionality attached to the 2-oxo acid portion of the substrate. Other enzymes that use substrates structurally similar to oxalyl-CoA decarboxylase include hydroxypyruvate decarboxylase and 3-phosphonopyruvate decarboxylase (FIG. 16, Scheme 2, Reaction D and E) and may also be used in the biosynthesis pathways disclosed herein.

Decarboxylases that decarboxylate alpha-keto-acids and are linked to an aromatic substituent may also be used in the metabolic pathways and microorganisms disclosed herein, such as benzoylformate decarboxylase encoded by the mdlC gene in P. putida ATCC 12633. The amino acid sequence of benzoylformate decarboxylase encoded by mdlC is shown in SEQ ID NO: 47. MdlC is an enzyme in the mandelate pathway and catalyzes the decarboxylation of benzoylformate to form benzaldehyde (FIG. 16, Scheme 2, Reaction K), however, it has been successfully changed to an active pyruvate decarboxylase by site-directed mutagenesis of identified residues. Alternatively, the gene ipdC encoding indolepyruvate decarboxylase that catalyses the reaction of indole-3-pyruvate to form indole acetaldehyde may be used in the metabolic pathways and microorganisms. Indolepyruvate decarboxylase has been reported in Pantoes agglomerans and Enterobacter cloacae. Perhaps the most promiscuous aromatic 2-ketoacid decarboxylase is the Aro10 encoded decarboxylase from Saccharomyces cerevisiae. Although yeast such as S. cerevisiae cannot use amino acids as a source of carbon for growth and metabolism, amino acids are still degraded as a source of ammonia and as sinks of reducing equivalents. For example, phenylalanine is converted by S. cerevisiae to phenylpyruvate and ammonia. Phenylpyruvate is then decarboxylated to phenylacetaldehyde (FIG. 16, Scheme 2, Reaction L), which is then further degraded into phenylethanol or phenylacetic acid. S. cerevisiae uses this pathway (Ehrlich pathway) to degrade methione, leucine, isoleucine and valine. The corresponding decarboxylase activity has been demonstrated to be catalyzed by Aro10. Reactions that are shown to be catalyzed by Aro 10 are summarized (FIG. 16, Scheme 2, Reactions F-J and L-M).

Returning to FIG. 1, as shown, the dehydrogenation step to convert adipate semialdehyde to adipate may be catalyzed by a ChnE enzyme or a homolog of the ChnE enzyme. ChnE is an NADP-linked 6-oxohexanoate dehydrogenase enzyme (i.e., adipate semialdehyde dehydrogenase) and has been to shown to catalyze the dehydrogenation of the 6-oxohexanoate to adipate in the cyclohexanol degradation pathway in Acinetobacter sp. (see Iwaki et al., Appl. Environ. Microbiol. 1999, 65(11): 5158-5162).

In some examples, adipate semialdehyde dehydrogenase may be ChnE from Acinetobacter sp. NCIMB9871 and have an amino acid sequence according to SEQ ID NO: 20. In other examples, the adipate semialdehyde dehydrogenase may be ChnE from Acinetobacter sp. NCIMB9871 and be encoded by the nucleotide sequence of SEQ ID NO: 21, which is codon-optimized for expression in E. coli. In another example, alpha-ketoglutaric semialdehyde dehydrogenase (EC 1.2.1.26, for example AraE) converts adipate semialdehyde into adipate.

In addition to the production of adipic acid, we also provide engineered pathways for the production of other difunctional alkanes of interest. Particularly, aspects of this disclosure relate to the production of amino caproic acid (a stable precursor of caprolactam acid), hexamethylene diamine and 6-hydroxyhexanoate. Other suitable biosynthesis pathways for preparing C5-C8 difunctional alkanes using alpha-ketoacid as a precursor include those disclosed in U.S. Pat. No. 8,133,704, incorporated herein by reference it its entirety. For example, rather than conversion of adipate semialdehyde to adipic acid, a biosynthesis pathway may be engineered to include an amino-transferase enzyme step for conversion of adipate semialdehyde to amino caproic acid.

Alternatively or additionally, the biosynthesis pathway may be engineered for conversion of 2-aminopimelate produced from alpha-ketopimelate by 2-aminotransferase and to hexamethylenediamine by combining enzymes or homologous enzymes characterized in the Lysine biosynthetic pathway. Specifically, the biosynthesis pathway may convert 2-aminopimelate to 2-amino-7-oxoheptanoate (or 2 aminopimelate 7 semialdehyde) as catalyzed for example by an amino adipate reductase or homolog enzyme (e.g. Sc-Lys2, EC 1.2.1.31); convert 2-amino-7-oxoheptanoate to 2,7-diaminoheptanoate as catalyzed for example by a saccharopine dehydrogenase (e.g. Sc-Lys9, EC 1.5.1.10 or Sc-Lys1, EC 1.5.1.7); then convert 2,7-diaminoheptanoate to hexamethylene diamine as catalyzed for example by a Lysine decarboxylase or an ornithine decarboxylase.

The microorganisms and methods of this disclosure can be used advantageously in connection with the engineered biosynthesis pathways discussed above. We provide microorganisms and methods for increasing the production of difunctional alkanes in host cells that produce difunctional alkanes from alpha-keto acids, particularly alpha-ketoglutarate. In one aspect, we provide methods to increase homocitrate production relative to wild-type by increasing alpha-ketoglutarate flux. Increased production of homocitrate may contribute to an increased availability of homocitrate as substrate for additional alpha-keto elongation reactions and conversion of alpha-keto acid to a difunctional alkane.

One suitable method of increasing alpha-ketoglutarate flux is alteration of the expression and/or activity of the proteins encoded by chromosomal sucA (E.C. 1.2.4.2.) and aceA genes (E.C. 4.1.3.1.). The amino acid sequence of an exemplary E. coli sucA protein is shown in SEQ ID NO: 48 and the amino acid sequence of an exemplary E. coli aceA protein is shown in SEQ ID NO: 49.

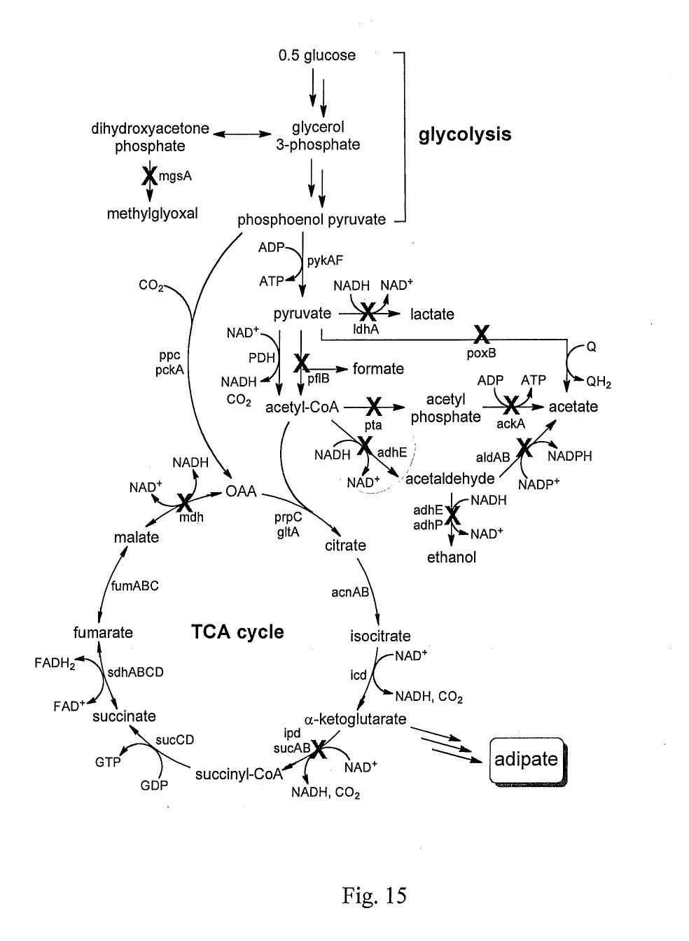

The sucAB gene encodes an alpha-ketoglutarate dehydrogenase complex that is part of the TCA cycle and catalyzes the oxidative decarboxylation of alpha-ketoglutarate into succinyl-CoA by a series of reactions, as shown in FIG. 15. Deficiency in alpha-ketoglutarate dehydrogenase activity has been reported to produce L-glutamic acid at a higher level than wild-type and a single sucA gene knockout in E. coli BW25113 strain has been found to result in a 5.5-fold increase (from 0.25 to 1.4 mM) in intracellular alpha-ketoglutarate concentration. See, U.S. Pat. No. 5,378,616; Li, M.; Ho, P. Y.; Yao, S.; Shimizu, K. Biochem. Eng. J. 2006, 30, 286. Accordingly, a deficiency in alpha-ketoglutarate dehydrogenase activity, such as by knocking-out or attenuating the expression of the sucA gene or decreasing the activity of the alpha-ketoglutarate dehydrogenase protein, will enhance production of homocitrate due to increased intracellular availability of alpha-ketoglutarate by preventing or reducing conversion of alpha-ketoglutarate into succinyl-CoA. However, due to the disruption of the TCA cycle, mutant E. coli lacking alpha-ketoglutarate dehydrogenase activity requires succinate for aerobic growth on glucose minimal medium (Guest, J. R.; Herbert, A. A. Mol. Gen. Genet. 2969, 105, 182).

It has also be shown that the sucA mutant down-regulated global regulator genes such as fadR and iclR. Li, supra. The consequence of this down regulation is the activation of the glyoxylate pathway by enhanced expression of aceA gene encoding isocitrate lyase (EC 4.1.3.1).

Isocitrate lyase is an enzyme in the glyoxylate cycle that catalyzes the cleavage of isocitrate to succinate and glyoxylate. The glyoxylate cycle is used by bacteria, fungi, and plants and is involved in the conversion of acetyl-CoA to succinate for the synthesis of carbohydrates. In microorganisms, the glyoxylate cycle allows cells to utilize simple carbon compounds as a carbon source when complex sources such as glucose are not available. In this alternative pathway, malate synthase and isocitrate lyase allow the metabolic pathways to bypasses the two decarboxylation steps of the tricarboxylic acid cycle (TCA cycle). Accordingly, expressing or overexpressing isocitrate lyase (aceA) may assist in compensating for any loss of succinate production or other TCA-cycle intermediates resulting from a deficiency in alpha-ketoglutarate dehydrogenase activity.

Accordingly, we provide modified microorganisms having a deficiency compared to a parent or wild-type cell in alpha-ketoglutarate dehydrogenase activity and/or not expressing alpha-ketoglutarate dehydrogenase. Additionally, we provide microorganisms having an increase in activity of isocitrate lyase, such as by expressing additional copy numbers or overexpressing isocitrate lyase compared to a parent or wild-type cell.

Alteration of the expression or activity of the proteins may be achieved, for example, by deletion, mutation, increase in copy number or other alteration of the chromosomal sucA and aceA genes. These modifications will result in a microorganism having a deficiency in catalyzing the oxidative decarboxylation of alpha-ketoglutarate into succinyl-CoA compared to a wild-type or parent cell. Additionally, by utilizing isocitrate lyase and the glyoxylate pathway, the cell can produce succinate as a substrate for the TCA-cycle.

‘Knock-out’ and ‘knock-in’ of genes in E. coli may be performed using λ-mediated recombination E. coli recombineering technology described in U.S. Pat. Nos. 6,509,156; 6,355,412 and U.S. application Ser. No. 09/350,830, which are each incorporated herein by reference. In λ-mediated recombination, also referred to as RED/ET® Recombination (GENE BRIDGES), target DNA molecules, such as chromosomal DNA, in strains of E. coli expressing phage-derived protein pairs may be altered by homologous recombination. The phage-derived protein pairs include a 5′->3′ exonuclease and DNA annealing proteins. For example, RecE and Reda may be the 5′->3′ exonucleases, and RecT and Redβ may be the DNA annealing proteins. A functional interaction between the 5′->3′ exonuclease and DNA annealing proteins catalyses a homologous recombination reaction. Recombination occurs at portion of the DNA, called homology regions, which are shared by the two molecules that recombine and can be at any position on a target molecule.

For knock-in or knock-out of the chromosomal sucA, aceA and other genes, PCR primers may be based on 50-60 nucleotide homologous sequence for the gene to be deleted and 20 nucleotides for the priming site on resistance gene marker templates. PCR product can be introduced into E. coli transformed with plasmid pRedET, sold by GENE BRIDGES, by electroporation. Plasmid pRedET encodes for λ-Red recombinase. Strains that are resistant to antibiotics are first selected on LB agar plates, followed by PCR confirmation of the genomic region.

Suitable techniques include introducing the knockout into E. coli, such as but not limited to BW25113, and then P1 transduction of the marker-linked knockout into desired biocatalyst. Alternatively, a commercially available E. coli strain having a single gene mutation, such as those available from the Keio Collection, may be used. However, the combination of λRed recombineering and P1 transduction is believed to more frequently provide a clean genomic background than use of an E. coli that has multiple FRT scars. The E. coli strain undergoing P1 transduction may carry a temperature sensitive pCP20 plasmid, which has a gene insert encoding FLP recombinase. In some cases, subsequent curing to remove the FRT-flanked drug markers may be necessary for construction of the multiple deletion final biocatalyst. Adverse polar effects may be associated with deletion mutations that are associated with drug markers, but may be removed upon removal of the drug markers.

In addition to altering the expression of alpha-ketoglutarate dehydrogenase (sucA) and/or isocitrate lyase (aceA) genes or the activity of the encoded enzymes, we provide microorganisms and methods of biasing the alpha-ketoglutarate flux to increase homocitrate production by knock out of the arcA gene. In E. coli the levels of numerous enzymes associated with aerobic metabolism are decreased during anaerobic growth. Part of the mechanism used by E. coli to respond to oxygen availability includes the activity of the Arc system, which is a two-component signal transduction system composed of ArcAB. The amino acid sequence of an exemplary arcA protein is shown in SEQ ID NO: 50. Modified ArcA represses the expression of major enzymes in the TCA cycle, including citrate synthase, aconitase and isocitrate dehydrogenase (Iuchi, S.; Lin, E. C. C. Pro. Natl. Acad. Sci. USA 1988, 85, 1888). Accordingly, a deficiency in the activity of the protein encoded by arcA, such as by knocking out arcA gene or attenuating the expression or activity of the arcAB protein complex, will avoid repression of TCA cycle enzymes, thereby resulting in the production of alpha-ketoglutarate through TCA. Increase in alpha-ketoglutarate is expected to drive the metabolism towards production of difunctional alkanes, such as adipic acid and others.

Accordingly, we provide microorganisms and biosynthetic pathways modified to eliminate the arcA gene or reduce expression of the gene. Additionally, we also provide microorganisms that are modified to reduce or alter the activity of the arcAB protein complex, such as by mutation of amino-acids or polypeptides involved in catalysis or protein folding using techniques known to one skilled in the art. These microorganisms may, therefore, have a deficiency in arcA activity.

We also provide microorganisms having increased homocitrate production by modification to include a NADH-insensitive citrate lyase enzyme. The amino acid sequence of an exemplary E. coli NADH-insensitive citrate lyase is shown in SEQ ID NO: 51. Citrate lyase is involved in the TCA cycle and, thus, plays a role in the production and consumption of compounds in the pathway and regulates the flow of carbon towards alpha-ketoglutarate. However, depending on growth conditions, reduction in citrate synthase activity can reduce the carbon flux away from alpha-ketoglutarate. Accordingly, in order to increase production of alpha-ketoglutarate, it would be desirable to avoid the inhibition of citrate synthase activity.

However, in E. coli, native citrate synthase activity (GltA) is known to be inhibited by high NADH concentration in the cell. As a total of 11 NADH is generated for each adipic acid produced using the biosynthetic pathway discussed herein, the produced NADH constrains the activity of citrate synthase and the flux towards alpha-ketoglutarate. Accordingly, recruitment of an NADH-insensitive citrate synthase may reduce or avoid inhibition of citrate synthase activity. Furthermore, recruiting NADH insensitive citrate synthase for adipic acid biosynthesis pathways is believed to increase alpha-ketoglutarate availability, therefore might also increase homocitrate production.

A suitable NADH-insensitive citrate synthase may be derived from gram-positive bacteria. Unlike most gram-negative bacteria, gram-positive counterparts are usually insensitive to NADH. For example, expression of Bacillus subtilis citrate synthase citZ in E. coli improves xylose fermentation to ethanol and may be used in the biosynthesis pathways disclosed herein (Underwood, S. A.; Buszko, M. L.; Shanmugam, K. T.; Ingram, L. O. Appl. Environ. Microbiol. 2002, 68, 1071). The amino acid sequence of the Bacillus subtilis citrate synthase citZ is shown in SEQ ID NO: 52. Alternatively, the native citrate synthase enzyme can be modified to reduce sensitivity to NADH by techniques known in the art. For example, amino acid R163L mutation in citrate synthase gltA was reported to reduce inhibition by NADH (Pereira, D. S. Donald, L. J.; Hosfield, D. J.; Duckworth, H. W. J. Biol. Chem. 1994, 269, 412).

Accordingly, we provide microorganisms modified to include either or both an NADH-insensitive citrate synthase derived from a gram-positive bacteria or a citrate synthase modified to reduce sensitivity, such as by the R163L amino acid mutation. An NADH-insensitive citrate synthase may be introduced to supplement the native citrate synthase or, alternatively, the native citrate synthase may be deleted and/or rendered inoperable such that the NADH-insensitive citrate synthase replaces the native citrate synthase.

We further provide methods and microorganisms for improving alpha-ketopimelate and adipic acid production by increasing acetyl-CoA flux. Acetyl-CoA is necessary for citrate synthase to convert oxaloacetate into citrate. Thus, the availability of acetyl-CoA may serve as a rate limiting factor in the TCA cycle. Accordingly, modifications that increase the cellular availability of acetyl-CoA may help feed the TCA cycle, thereby increasing the production of alpha-ketoglutarate and adipic acid.

A suitable method of increasing acetyl-CoA flux may include overexpression of acetyl-CoA synthetase. Acetyl-CoA synthetase (EC 6.2.1.1) is an enzyme involved the reversible conversion of acetate and CoA to Pyrophosphate acetyl-CoA. The amino acid sequence of an exemplary acetyl-CoA synthetase is shown in SEQ ID NO: 53.

Under aerobic growth conditions, E. coli uses glucose as a carbon source and produces a significant amount of acetate. Not only is a high level of acetate accumulation harmful to cell growth, but the acetate pathway can also consume a portion of the cellular acetyl-CoA. Accordingly, it would be desirable to reduce the production of acetate. A suitable method for reducing the production of acetate is to knock-out or attenuate the enzymes in the primary acetate pathway, such as pta and ackA. However, alterations or mutations in the primary acetate producing pathway, such as pta and ackA knockouts, are known to reduce cell growth. Accordingly, we provide modified microorganisms overexpressing acetyl-CoA synthetase to increase the cellular availability of acetyl-CoA by reducing conversion of acetyl-CoA to acetate. By increasing the acetyl-CoA intracellular availability, overexpression of acetyl-CoA synthetase is expected to direct carbon flux towards producing difunctional hexanes in the proposed pathway.

Additionally or alternatively, pyruvate dehydrogenase can be modified to reduce or eliminate feedback sensitivity, thereby increasing acetyl-CoA availability for alpha-ketoglutarate and adipic acid production. The amino acid sequence of an exemplary E. coli pyruvate dehydrogenase 1pd is shown in SEQ ID NO: 54. E. coli pyruvate dehydrogenase catalyzes the formation of acetyl-CoA using NAD+ as a cofactor, but may have low activity under oxygen-limited or anaerobic conditions due to the higher NADH/NAD⁺ ratio. One explanation for this inactivity is that the E3 subunit of pyruvate dehydrogenase complex (lpd) is inhibited by NADH. However, the amino acid E354K mutation in Lpd has been shown to be significantly less sensitive to NADH inhibition than native Lpd (Kim, Y.; Ingram, L. O.; Shanmugam, K. T. J. Bacteriol. 2008, 190, 3851). Furthermore, K. pneumoniae Lpd has >90% DNA identity compared to the E. coli, but is known to function anaerobically (Menzel, K.; Zeng, A. P.; Deckwer, W. D. J. Biotechnol. 1997, 56, 135).

Accordingly, we provide a modified difunctional alkane-producing microorganism having a pyruvate dehydrogenase modified to reduce or eliminate feedback sensitivity. For example, the pyruvate dehydrogenase may have a E354K point mutation and/or the native pyruvate dehydrogenase can be replaced with or supplemented by a pyruvate dehydrogenase that functions under anaerobic conditions, such as K. pneumoniae Lpd. The amino acid sequence of an exemplary K. pneumoniae pyruvate dehydrogenase lpd is shown in SEQ ID NO: 55.

In addition to modifying the expression and the activity of enzymes that are directly metabolically tied to the difunctional alkane synthetic pathway, we also provide microorganisms modified to knock-out genes or otherwise reduce the activity of enzymes that are known to produce unwanted byproducts. By reducing the production of unwanted by-products, carbon flux can be directed to increase adipic acid production yield. Additionally, eliminating or reducing byproducts formation simplifies associated downstream processing and reduces cost and energy.

Genes that may be knocked-out to reduce by-product formation include but are not limited to poxB, pflB, pta, ackA, adhE, aldAB, adhE, adhP, mgsA and ldhA. Each of these genes encodes an enzyme that catalyzes a reaction that diverts carbon away from the production of adipic acid and towards unwanted by-products. For example, ldhA encoding lactate dehydrogenase results in lactate production. The gene mgsA encoding methylglyoxal synthase catalyzes the conversion of dihydroxyacetone phosphate into methylglyoxal, which is also toxic to cells. Inactivation of the ldhA and mgsA genes will yield positive effects to the process, without creating a severe metabolic burden for aerobic/microaerobic cultivation.

Additionally or alternatively, the microorganism can be modified to include knockouts of poxB encoding pyruvate oxidase, pflB encoding pyruvate-formate lyase, pta encoding phosphotransacetylase, ackA encoding acetate kinase and aldB encoding aldehyde dehydrogenase or otherwise have a deficiency in the activity of expression of these enzymes. The enzymes encoded by these genes tend to convert acetyl-CoA and alpha-ketoglutarate intermediates into a variety of products for different reasons, including formate, acetyl phosphate, acetaldehyde, ethanol and acetate (see, FIG. 15). Diversion of acetyl-CoA to form formate, acetyl phosphate, acetaldehyde, ethanol and acetate results in less alpha-ketoglutarate to feed the keto-acid elongation pathway. Disruption of these genes will increase intracellular availability of the carbon building blocks for biosynthesis, ultimately increasing adipic acid yield from the pathway.

We further provide methods and microorganisms for improving difunctional alkane production including a microorganism having an alpha-ketopimelate decarboxylase with improved substrate specificity compared to wild-type. Keto-acid decarboxylases reported to be capable of using alpha-ketopimelate as substrate can be used in the biosynthesis pathways disclosed herein. FIG. 16 shows the substrates and reactions catalyzed by potentially suitable keto-acid decarboxylases. Preferably, keto-acid decarboxylases having improved substrate selectivity towards alpha-ketopimelate may be used. Additionally, suitable keto-acid decarboxylases may be obtained by directed evolution to improve substrate selectivity of known alpha-keto decarboxylases that are active towards alpha-ketopimelate.

Alternatively or additionally, the biosynthesis pathway is engineered for the production of 6-hydroxyhexanoate (6HH) from adipate semialdehyde, an intermediate of the adipic acid biosynthesis pathway described above. 6HH is a 6-carbon hydroxyalkanoate that can be circularized to caprolactone or directly polymerized to make polyester plastics (polyhydroxyalkanoate PHA). In some examples, adipate semialdehyde is converted to 6HH by simple hydrogenation and the reaction is catalyzed by an alcohol dehydrogenase (EC 1.1.1.1). This enzyme belongs to the family of oxidoreductases, specifically those acting on the CH—OH group of donor with NAD+ or NADP+ as acceptor. In some examples, a 6-hydroxyhexanoate dehydrogenase (EC 1.1.1.258) that catalyzes the following chemical reaction is used: 6-hydroxyhexanoate+NAD⁺ [symbol] 6-oxohexanoate+NADH+H⁺. Other alcohol dehydrogenases include but are not limited to adhA or adhB (from Z. mobilis), butanol dehydrogenase (from Clostridium acetobutylicum), propanediol oxidoreductase (from E. coli), and ADHIV alcohol dehydrogenase (from Saccharomyces).

One skilled in the art will appreciate that the biosynthetic pathways and microorganisms disclosed herein are further explained by the following representative and non-limiting examples.

### EXAMPLES

The materials used in the following Examples were as follows: Recombinant DNA manipulations generally followed methods described by Sambrook et al. Molecular Cloning: A Laboratory Manual, Third Edition, Sambrook and Russell, 2001, Cold Spring Harbor Laboratory Press, 3^{rd} Edition. Restriction enzymes were purchased from New England Biolabs (NEB). T4 DNA ligase was obtained from Invitrogen. FAST-LINK™ DNA Ligation Kit was obtained from Epicentre. Zymoclean Gel DNA Recovery Kit and DNA Clean & Concentrator Kit was obtained from Zymo Research Company. Maxi and Midi Plasmid Purification Kits were obtained from Qiagen. Antarctic phosphatase was obtained from NEB. Agarose (electrophoresis grade) was obtained from Invitrogen. TE buffer contained 10 mM Tris-HCl (pH 8.0) and 1 mM Na₂EDTA (pH 8.0). TAE buffer contained 40 mM Tris-acetate (pH 8.0) and 2 mM Na₂EDTA.

In Examples 1-7, restriction enzyme digests were performed in buffers provided by NEB. A typical restriction enzyme digest contained 0.8 μg of DNA in 8 μL of TE, 2 μL of restriction enzyme buffer (10× concentration), 1 μL of bovine serum albumin (0.1 mg/mL), 1 μL of restriction enzyme and 8 μL TE. Reactions were incubated at 37° C. for 1 h and analyzed by agarose gel electrophoresis. When DNA was required for cloning experiments, the digest was terminated by heating at 70° C. for 15 min followed by extraction of the DNA using Zymoclean gel DNA recovery kit.

The concentration of DNA in the sample was determined as follows. An aliquot (10 μL) of DNA was diluted to 1 mL in TE and the absorbance at 260 nm was measured relative to the absorbance of TE. The DNA concentration was calculated based on the fact that the absorbance at 260 nm of 50 μg/mL of double stranded DNA is 1.0.

Agarose gel typically contained 0.7% agarose (w/v) in TAE buffer, Ethidium bromide (0.5 μg/ml) was added to the agarose to allow visualization of DNA fragments under a UV lamp. Agarose gel was run in TAE buffer. The size of the DNA fragments were determined using two sets of 1 kb Plus DNA Ladder obtained from Invitrogen.

Table 2 shows the primer sequences used to generate plasmids expressing enzymes in the keto-extention pathway in the following Examples.

TABLE 2
Oligonucletides for Cloning Genes in the Keto-Extension Pathway

| | | |
|---|---|---|
| KL014 | (SEQ ID NO: 22) | CACCCGGGAGAAGGAGATATACATATGACCCTG |
| KL015 | (SEQ ID NO: 23) | GCATCGATTATGCGGCCGTGTACAATACG |
| KL021 | (SEQ ID NO: 24) | CCGGATCCTACCATGGCGTCAGTCATTATCGAT |
| KL022 | (SEQ ID NO: 25) | CTAGAAGCTTCCTAAAGCAGGTTAGGCCATACCGCCTGCG |
| KL023 | (SEQ ID NO: 26) | GCGTATAATATTTGCCCATTGTGAAAACGGGGGCGAA |
| KL024 | (SEQ ID NO: 27) | GTCTTTCATTGCCATACGAAATTCCGGATGAGCATTC |
| KL025 | (SEQ ID NO: 28) | CGACCCCGGGAAGCTTCGATGATAAGCTGTCAAACATGAGA |
| KL026 | (SEQ ID NO: 29) | CGATGGATCCGATATCTCACTTATTCAGGCGTAGCACCAGG |
| KL029 | (SEQ ID NO: 30) | CGAGGATCCTCATGATTATTAAAGGCCGTGCCCACA |
| KL044 | (SEQ ID NO: 31) | TCTAGATATCAAGCTTTCTAGAAACGAAAGGCCCAGTCTTT |
| KL045 | (SEQ ID NO: 32) | ATCCGATATCGGATCCGAGCTCCATGCACAGTGAAATCATA |
| KL051 | (SEQ ID NO: 33) | GCCGCGGATCCCTCGAGTTAATCCAGTTTATTGGTAATATAG |

Table 3 shows the primer sequences used to generate plasmids expressing α-ketopimelate decarboxylase enzymes

TABLE 3
Oligonucletides for Cloning genes encoding α-ketopimelate
decarboxylase

| | | |
|---|---|---|
| KL028 | (SEQ ID NO: 34) | ATATCCTTAAGCTCGAGCAGCTGGCGGCCGCTTAT |
| KL031 | (SEQ ID NO: 35) | CGCTGAATTCACATGTATACCGTGGGCGACTACCTGC |
| KL032 | (SEQ ID NO: 36) | CGTGCGGCCGCCTCGAGTTACGATTTATTTTGTTCAGCGAAC |
| NS001 | (SEQ ID NO: 37) | CGTTCAGGAATTGGATCCTATACCGTGGGCGACTACCTGC |
| NS002 | (SEQ ID NO: 38) | CGTTCAGGAATTGGATCCTACACCGTGGGCGACTATCTGC |

### Example 1

Cloning of Plasmid pBA006

Plasmid pETDuet-nifV-aksF_Mb was constructed from base vector pETDuet1 (Novagen) engineered to include the E. coli codon-optimized homocitrate synthase (nifV) from Azotobacter vinelandii encoded by the sequence shown in SEQ ID NO: 2 and homoisocitrate dehydrogenase (aksF_Mb) from Methanosarcina barkerii shown in SEQ ID NO: 5.

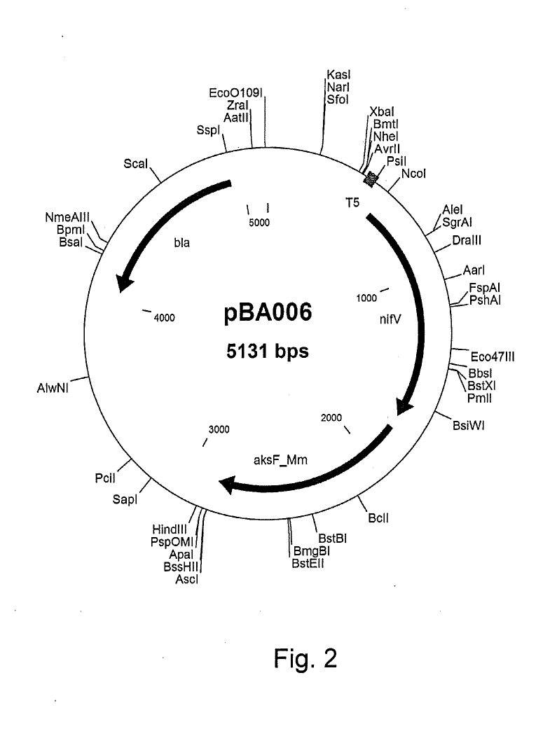

Plasmid pBA001 was constructed from base vector pUC57 to include the T5 promoter region according to SEQ ID NO: 15 and the E. coli codon-optimized homoisocitrate dehydrogenase (aksF_Mm) from Methanococcus maripaludis shown in SEQ ID NO: 6. The DNA fragment containing the nifV ORF was amplified from pETDuet-nifV-aksF_Mb by PCR using primers KL021 (SEQ ID NO: 24) and KL022 (SEQ ID NO: 25). The resulting 1.2 kb DNA was digested with NcoI and EcoNI. The 4.0 kb DNA fragment containing the pUC57 plasmid backbone, T5 promoter region, and aksF_Mm genes was obtained by restriction enzyme digestion of pBA001 using NcoI and EcoNI. The two DNA fragments were ligated to produce plasmid pBA006, as shown by schematic diagram in FIG. 2.

### Example 2

Cloning of Plasmid pBA008

Plasmid pBA002 was constructed from base vector pUC57 to include the T5 promoter region according to SEQ ID NO: 15 and the E. coli codon-optimized homoaconitase (aksDE_Mm) from Methanococcus maripaludis according to SEQ ID NOs: 9 and 10.

Plasmid pACYC184D was generated from pACYC184 by QuikChange site-directed mutagenesis (Stratagene) using primers KL023 (SEQ ID NO: 26) and KL024 (SEQ ID NO: 27) to remove restriction enzyme sites NcoI and EcoRI.

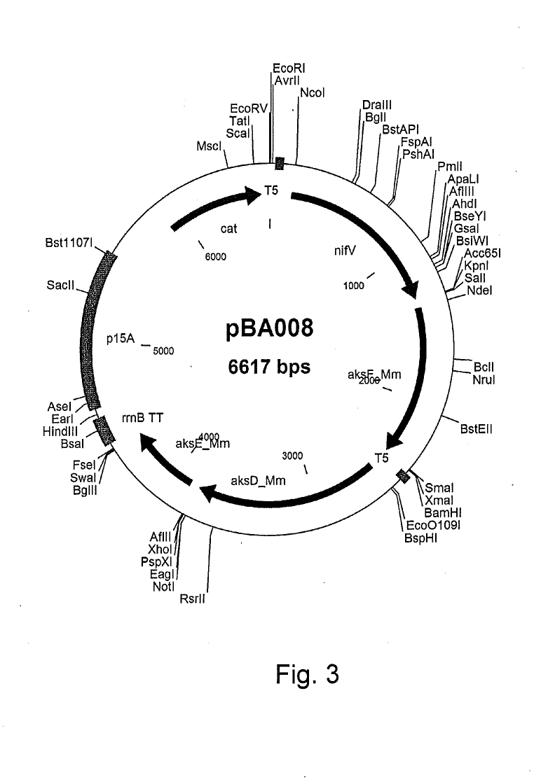

The 2.2 kb DNA fragment containing a T5 promoter region and aksDE_Mm genes was amplified by PCR using primers KL044 (SEQ ID NO: 31) and KL045 (SEQ ID NO: 32) from pBA002. The resulting fragment was digested with BamHI and HindIII. The 2.0 kb DNA fragment containing the p15A replication origin and the chloramphenicol resistance cassette was amplified from pACYC184D by PCR using primers KL025 (SEQ ID NO: 28) and KL026 (SEQ ID NO: 29). This fragment was digested by EcoRV and HindIII. The 2.4 kb DNA fragment containing the T5 promoter region, nifV and aksF_Mm was excised from pBA006 using BamHI and EcoRV. The three fragments were used in a three piece ligation reaction to produce plasmid pBA008, as shown by schematic diagram in FIG. 3

### Example 3

Cloning of Plasmid pBA019

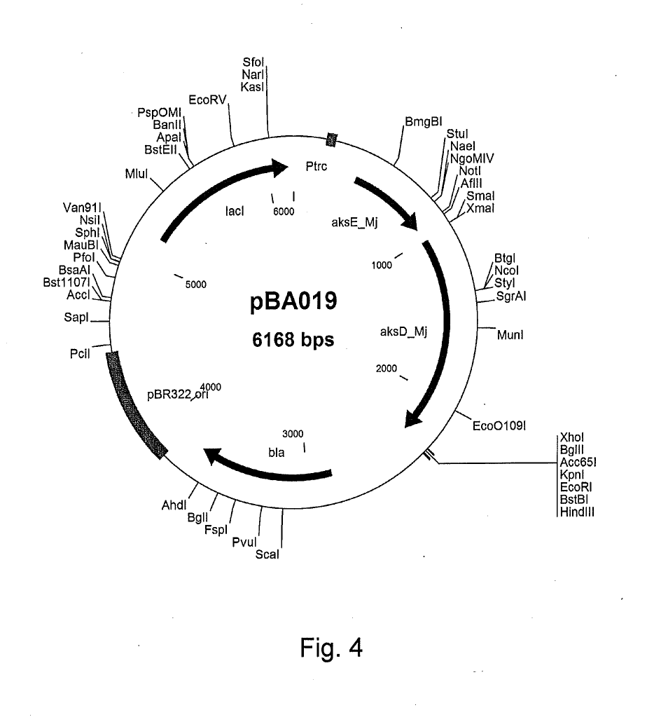

Plasmid pCDFDuet-aksED_Mj was constructed from base vector pCDFDuet1 (Novagen) to include the E. coli codon-optimized homoaconitase (aksED_Mj) from Methanocaldococcus jannaschii shown in SEQ ID NOs: 14 and 13. A DNA fragment was amplified from pCDFDuet-aksED_Mj by PCR using primers KL014 (SEQ ID NO: 22) and KL015 (SEQ ID NO: 23). Religation of the resulting 5.3 kb fragment produces pBA016. In this resulting plasmid, transcription of aksED_Mj is driven by a single T7 promoter. A 1.9 kb DNA fragment containing the aksED_Mj ORFs were amplified by PCR from pBA016 using primers KL029 (SEQ ID NO: 30) and KL051 (SEQ ID NO: 33). The resulting fragment was digested with BspHI and XhoI. Ligation with pTrcHisA (Invitrogen), which was pre-digested with NcoI and XhoI produced plasmid pBA019, as shown by schematic diagram in FIG. 4.

### Example 4

Cloning of Plasmid pBA029

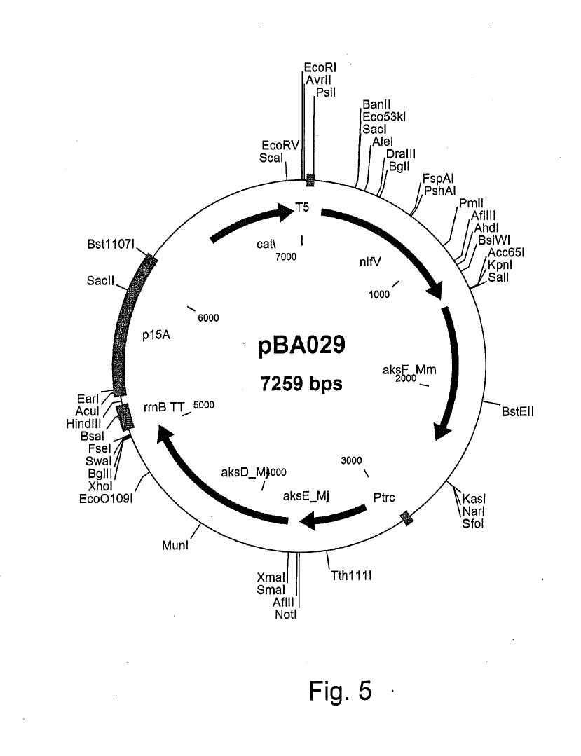

A 2.6 kb DNA fragment containing the trc promoter region and the aksED_Mj ORFs was excised from pBA019 using EcoRV and BglII. The 2.6 kb DNA fragement was ligated with another DNA fragment of plasmid pBA008, which was pre-digested with SmaI and BglII, to produce plasmid pBA029, as shown by schematic diagram in FIG. 5.

### Example 5

Cloning of Plasmid pBA021

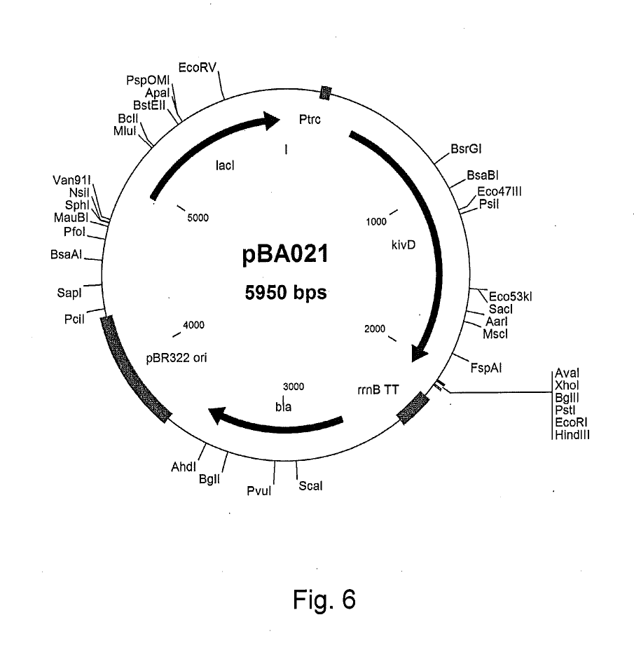

Plasmid pET21a-kivD was constructed from base vector pET21a (Novagen) to include the E. coli codon-optimized ketoisovalerate decarboxylase gene (kivD) from Lactococcus lactis KF147 as shown in SEQ ID NO: 17. The 1.6 kb kivD ORF was amplified by PCR using primers KL031 (SEQ ID NO: 35) and KL032 (SEQ ID NO: 36). The resulting DNA fragment was digested with PciI and XhoI. This fragment was ligated with the linearized pTrcHisA vector, which had been digested with NcoI and XhoI to produce plasmid pBA021, as shown by schematic diagram in FIG. 6.

### Example 6

Cloning of Plasmids pBA049 and pBA050

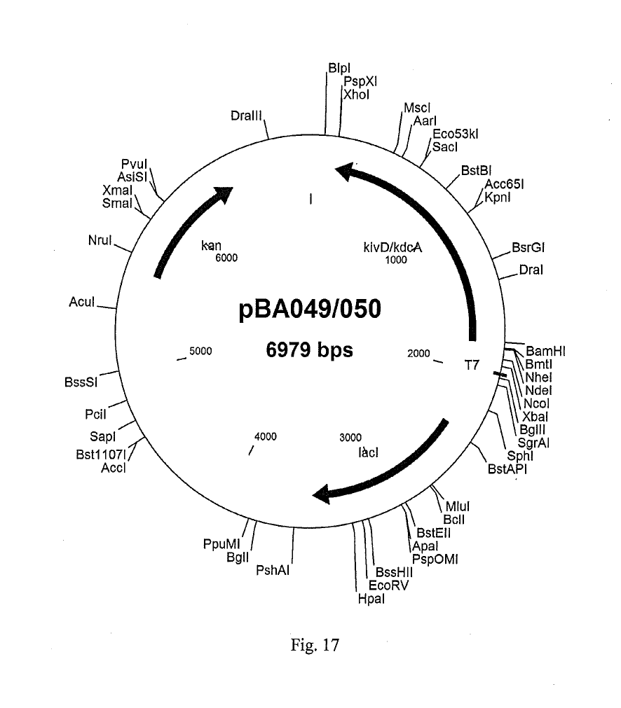

Plasmid pBA005 was constructed from base vector pUC57 to include the E. coli codon-optimized branched-chain ketoacid decarboxylase (kdcA) from Lactococcus lactis B 1157 as shown in SEQ ID NO: 19. The 1.6 kb kivD and kdcA ORFs were amplified from pBA021 and pBA005 by PCR using primer pairs NS001/KL032 (SEQ ID NO: 37/SEQ ID NO: 36) and NS002/KL028 (SEQ ID NO: 38/SEQ ID NO: 34), respectively. The resulting DNA fragments were digested individually with BamHI and XhoI. These fragments were ligated with the linearized pET28a vector, which had been digested with BamHI and XhoI to produce plasmids pBA049 and pBA050. pBA049 included kivD and pBA5050 included kdcA, as shown in FIG. 17.

pET28a is a commercial vector obtained from Novagen. It carries an N-terminal His•Tag®/thrombin/T7•Tag® configuration plus an optional C-terminal His•Tag sequence. The transcription of gene is driven by a phage T7 promoter.

### Example 7

Cloning of Plasmid pBA042

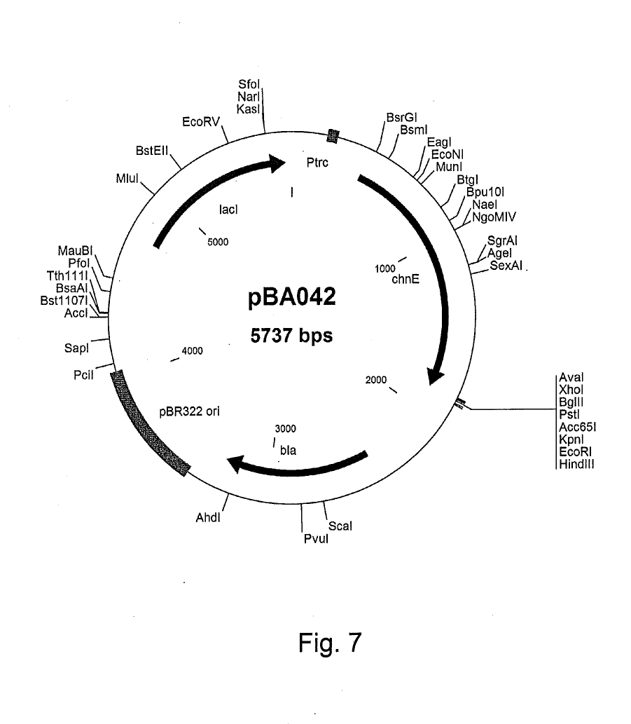

Plasmid pBA032 was constructed from base vector pUC57 to include the E. coli codon-optimized adipate semialdehyde dehydrogenase gene (chnE) gene from Acinetobacter sp. NCIMB9871 as shown in SEQ ID NO: 21. The 1.4 kb chnE ORF was excised from pBA032 using BspHI and XhoI. This fragment was ligated with the linearized pTrcHisA vector, which had been digested with NcoI and XhoI to produce plasmid pBA042, as shown by schematic diagram in FIG. 7.

### Example 8

Circular plasmid DNA molecules were introduced into target E. coli cells by chemical transformation or electroporation. For chemical transformation, cells were grown to mid-log growth phase, as determined by the optical density at 600 nm (0.5-0.8). The cells were harvested, washed and finally treated with CaCl₂. To chemically transform these E. coli cells, purified plasmid DNA was allowed to mix with the cell suspension in a microcentrifuge tube on ice. A heat shock was applied to the mixture and followed by a 30-60 min recovery incubation in rich culture medium. For electroporation, E. coli cells grown to mid-log growth phase were washed with water several times and finally resuspended into 10% glycerol solution. To electroporate DNA into these cells, a mixture of cells and DNA was pipetted into a disposable plastic cuvette containing electrodes. A short electric pulse was then applied to the cells which in turn causing small holes in the membrane where DNA could enter. The cell suspension was then incubated with rich liquid medium followed by plating on solid agar plates. Detailed protocol could be obtained in Molecular Cloning: A Laboratory Manual, Third Edition, Sambrook and Russell, 2001, Cold Spring Harbor Laboratory Press, 3^{rd} Edition

E. coli cells of the BL21 strain were transformed with the plasmids previously described in Examples 1-7. BL21 is a strain of E. coli having the genotype: B F⁻ dcm ompT hsdS(r_{B}-m_{B}-) gal λ.

Specifically, BL21 cells were separately transformed with plasmids pET28a (control), pBA049, pBA050, pBA032 and pBA042. Additionally, BL21 cells were transformed with both plasmids pBA029 and pBA021 to generate BA029.

### Example 9

Cell Lysis Method

E. coli cell culture was spun down by centrifugation at 4000 rpm. The cell-free supernatant was discarded and the cell pellet was collected. After being collected and resuspended in the proper resuspension buffer (50 mM phosphate buffer at pH 7.5), the cells were disrupted by chemical lysis using BUGBUSTER® reagent (Novagen). Cellular debris was removed from the lysate by centrifugation (48,000 g, 20 min, 4° C.). Protein was quantified using the Bradford dye-binding procedure. A standard curve was prepared using bovine serum albumin. Protein assay solution was purchased from Bio-Rad and used as described by the manufacturer.

### Example 10

ChnE Activity in BL21/pBA042 Crude Lysate

High-throughput in vitro adipate semialdehyde dehydrogenase activity was assayed in a 96-well plate format to verify expression and activity of adipate semialdehyde dehydrogenase (ChnE) in BL21 cells transformed with plasmid pBA042. The assay protocol was modified from a literature procedure (Iwaki H. Appl. Environ. Microbiol. 1999, 65, 5158).

A typical assay mixture was composed of 50 mM adipate semialdehyde methyl ester and 2 mM NAD (or 50 mM adipic acid and 2 mM NADH) in 50 mM potassium phosphate buffer at pH 7 to a total volume of 200 μL per well.

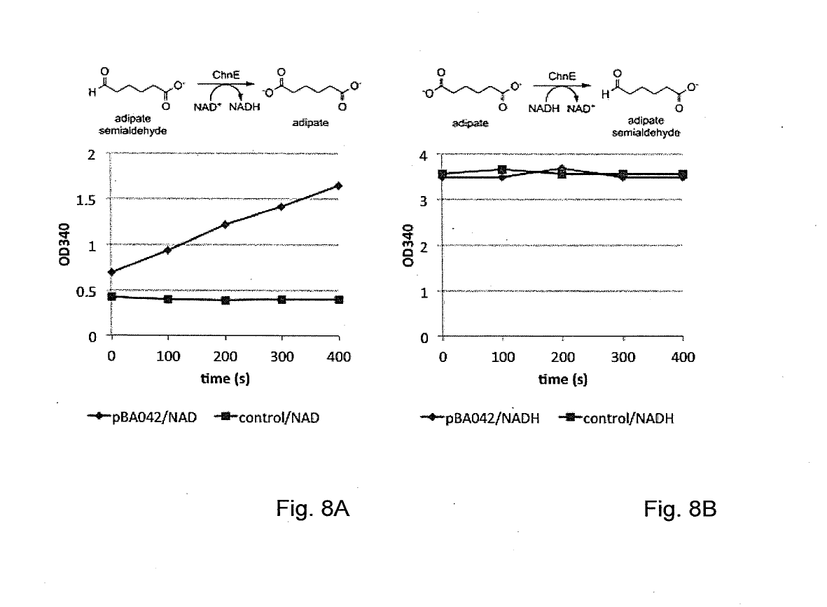

The assay was initiated by the addition of a 10 uL of cell lysate and was followed spectrophotometrically by monitoring formation of NADH at 340 nm. A unit of activity equals 1 μmol per min of NADH formed at 30° C. As shown in FIG. 8, BL21 control lysate showed negligible background activity when adipate semialdehyde methyl ester and NAD were used. Crude lysate of BL21/pBA042 showed activity at around 0.1 U/mg under the same conditions. It is important to note that the reverse reaction was at least 20-fold slower when adipic acid and NADH were used in the reaction mixture, thus indicating that the reaction is biased toward the formation of adipic acid.

### Example 11

SDS-PAGE Analysis of Decarboxylase and Dehydrogenase Expression

SDS-PAGE was used to analyze protein expression in constructs BL21/pET28a (control), BL21/pBA049, BL21/pBA050, BL21/pBA032 and BL21/pBA042 (FIG. 2). Lanes 1 and 2 are samples of solution and the insoluble fraction of the pET28a construct, respectively. Lanes 3 and 4 are samples of solution and the insoluble fraction of the pBA049 construct, respectively. Lanes 5 and 6 are samples of solution and the insoluble fraction of the pBA050 construct, respectively. Lanes 7 and 8 are samples of solution and the insoluble fraction of the pBA032 construct, respectively. Lanes 9 and 10 are samples of solution and the insoluble fraction of the pBA042 construct, respectively.

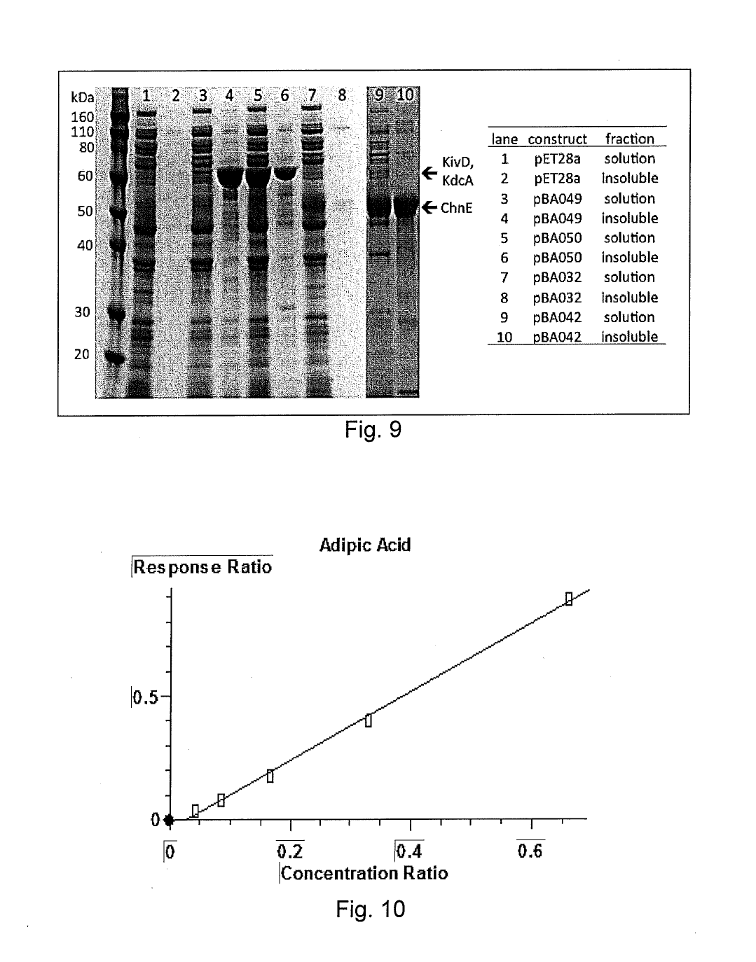

The molecular weight of the kivD and kdcA decarboxylase is 61 kDa, while the chnE gene encodes aldehyde dehydrogenase of 52 kDa. As shown in FIG. 9, proteins having the same molecular weight as KivD, KdcA and ChnE were successfully expressed.

### Example 12

GC/MS Method for Adipic Acid Quantification

Samples were prepared by transferring 1 mL of cell-free supernatant of samples taken from shake flasks or fermentation experiments to a microcentrifuge tube. Trichloroacetic acid (50 uL) was added to lower the sample pH. Ethyl acetate (0.5 mL×3) was used to extract the sample. Organic layers were collected, combined and dried under reduced pressure. The residue was then derivatized with N-tert-Butyldimethylsilyl-N-methyltrifluoroacetamide with 1% tert-Butyldimethylchlorosilane (MTBSTFA+t-BDMCS) silylation reagent (0.5 mL) and analyzed on the GC/MS. A calibration curve for adipic acid is shown in FIG. 10. FIG. 10 was obtained by plotting up data obtained from a GC/MS run. The y-axis is the area ratio of adipic acid to the internal standard. The x-axis is the concentration ratio of adipic acid to the internal standard.

Growth Medium

For the following Examples, Examples 13-15, the Growth Medium was prepared as follows:

All solutions were prepared in distilled, deionized water. LB medium (1 L) contained Bacto tryptone (i.e. enzymatic digest of casein) (10 g), Bacto yeast extract (i.e. water soluble portion of autolyzed yeast cell) (5 g), and NaCl (10 g). LB-glucose medium contained glucose (10 g), MgSO₄ (0.12 g), and thiamine hydrochloride (0.001 g) in 1 L of LB medium. LB-freeze buffer contained K₂HPO₄ (6.3 g), KH₂PO₄ (1.8 g), MgSO₄ (1.0 g), (NH4)2SO4 (0.9 g), sodium citrate dihydrate (0.5 g) and glycerol (44 mL) in 1 L of LB medium. M9 salts (1 L) contained Na₂HPO₄ (6 g), KH₂PO₄ (3 g), NH₄Cl (1 g), and NaCl (0.5 g). M9 minimal medium contained D-glucose (10 g), MgSO₄ (0.12 g), and thiamine hydrochloride (0.001 g) in 1 L of M9 salts. Antibiotics were added where appropriate to the following final concentrations: ampicillin (Ap), 50 μg/mL; chloramphenicol (Cm), 20 μg/mL; kanamycin (Kan), 50 μg/mL; tetracycline (Tc), 12.5 μg/mL. Stock solutions of antibiotics were prepared in water with the exceptions of chloramphenicol which was prepared in 95% ethanol and tetracycline which was prepared in 50% aqueous ethanol. Aqueous stock solutions of isopropyl-β-D-thiogalactopyranoside (IPTG) were prepared at various concentrations.

The standard fermentation medium (1 L) contained K₂HPO₄ (7.5 g), ammonium iron (III) citrate (0.3 g), citric acid monohydrate (2.1 g), and concentrated H₂SO₄ (1.2 mL). Fermentation medium was adjusted to pH 7.0 by addition of concentrated NH₄OH before autoclaving. The following supplements were added immediately prior to initiation of the fermentation: D-glucose, MgSO₄ (0.24 g), potassium and trace minerals including (NH₄)₆(Mo₇O₂₄).4H₂O (0.0037 g), ZnSO₄.7H₂O (0.0029 g), H₃BO₃ (0.0247 g), CuSO₄.5H₂O (0.0025 g), and MnCl₂.4H₂O (0.0158 g). IPTG stock solution was added as necessary (e.g., when optical density at 600 nm lies between 15-20) to the indicated final concentration. Glucose feed solution and MgSO₄ (1 M) solution were autoclaved separately. Glucose feed solution (650 g/L) was prepared by combining 300 g of glucose and 280 mL of H₂O, Solutions of trace minerals and IPTG were sterilized through 0.22-μm membranes. Antifoam (Sigma 204) was added to the fermentation broth as needed.

### Example 13

Shake Flask Experiments for Adipic Acid Production

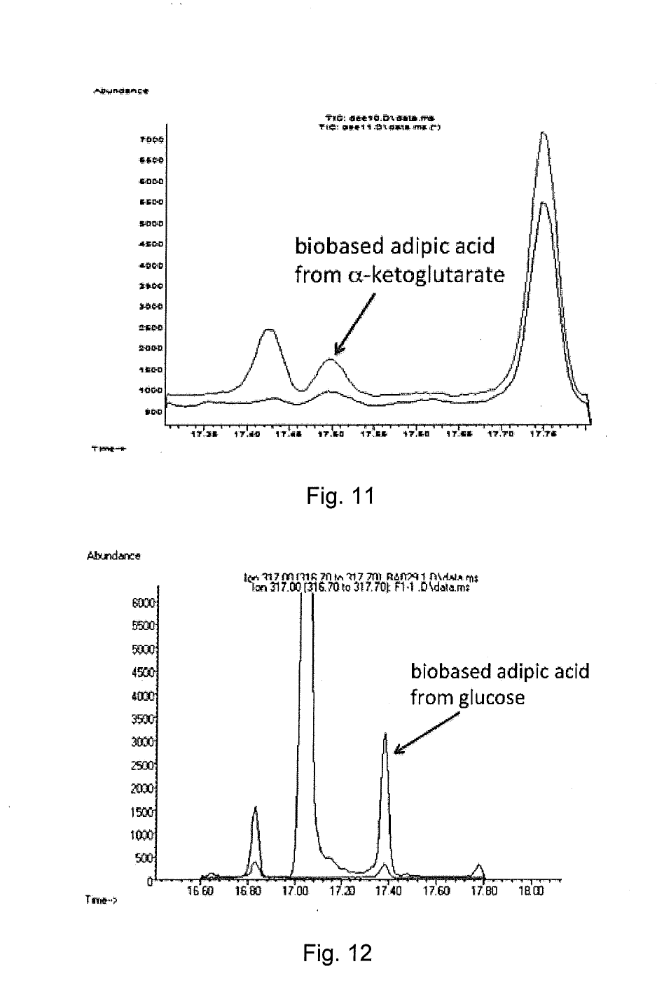

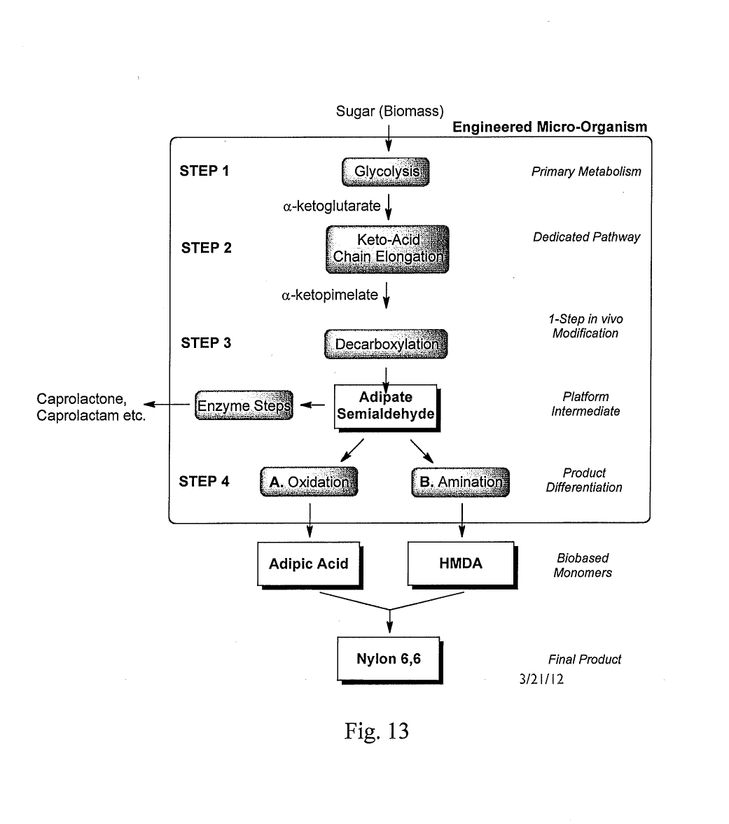

Seed inoculant was started by introducing a single colony of biocatalyst BA029 picked from a LB agar plate into 50 mL TB medium (1.2% w/v bacto Tryptone, 2.4% w/v Bacto Yeast Extract, 0.4% v/v glycerol, 0.017 M KH₂PO₄, 0.072 M K₂HPO₄). Culture was grown overnight at 37° C. with agitation at 250 rpm until they were turbid. A 2.5 mL aliquot of this culture was subsequently transferred to 50 mL of fresh TB medium. After culturing at 37° C. and 250 rpm for an additional 3 h, IPTG was added to a final concentration of 0.2 mM. The resulting culture was allowed to grow at 27° C. for 12 hours. Cells were harvested, washed twice with PBS medium, and resuspended in 0.5 original volume of M9 medium supplemented with α-ketoglutarate (2 g/L). The whole cell suspension was then incubated at 27° C. for 72 h. Samples were taken and analyzed by GC/MS. The results are shown in FIG. 11. Cell pellet was saved for SDS-PAGE analysis.

### Example 14

Adipic Acid Production with α-Ketoglutarate Spike-In

Compared to the control BL21 strain transformed with empty plasmids, E. coli BA029 produced adipic acid at a concentration of 11 ppm in shake flasks with α-ketoglutarate spiked-in (FIG. 11). Attempts to produce adipic acid using BA029 under shake flasks conditions were unsuccessful, although the proteins were expressed. It is believed that the amount of alpha-ketoglutarate inside cell may have been insufficient.

### Example 15

Cultivation of Adipic Acid Biocatalyst Under Fermentor-Controlled Conditions

Fed-batch fermentation was performed in a 2 L working capacity fermentor. Temperature, pH and dissolved oxygen were controlled by PID control loops. Temperature was maintained at 37° C. by temperature adjusted water flow through a jacket surrounding the fermentor vessel at the growth phase, and later adjusted to 27° C. when production phase started. The pH was maintained at 7.0 by the addition of 5 N KOH and 3 NH₃PO₄. Dissolved oxygen (DO) level was maintained at 20% of air saturation by adjusting air feed as well as agitation speed.

Inoculant was started by introducing a single colony of BA029 picked from an LB agar plate into 50 mL TB medium. The culture was grown at 37° C. with agitation at 250 rpm until the medium was turbid. Subsequently a 100 mL seed culture was transferred to fresh M9 glucose medium. After culturing at 37° C. and 250 rpm for an additional 10 h, an aliquot (50 mL) of the inoculant (OD600=6-8) was transferred into the fermentation vessel and the batch fermentation was initiated. The initial glucose concentration in the fermentation medium was about 40 g/L.

Cultivation under fermentor-controlled conditions was divided into two stages. In the first stage, the airflow was kept at 300 ccm and the impeller speed was increased from 100 to 1000 rpm to maintain the DO at 20%. Once the impeller speed reached its preset maximum at 1000 rpm, the mass flow controller started to maintain the DO by oxygen supplementation from 0 to 100% of pure O₂.

The initial batch of glucose was depleted in about 12 hours and glucose feed (650 g/L) was started to maintain glucose concentration in the vessel at 5-20 g/L. At OD600=20-25, IPTG stock solution was added to the culture medium to a final concentration of 0.2 mM. The temperature setting was decreased from 37 to 27° C. and the production stage (i.e., second stage) was initiated. Production stage fermentation was run for 48 hours and samples were removed to determine the cell density and quantify metabolites.

The adipic acid production was measured by GS/MS, and the results are shown in FIG. 12. As shown in FIG. 12, compared to the control BL21 strain transformed with empty plasmids, E. coli BA029 produced adipic acid from glucose at a concentration of 5 ppm under fermentor-controlled conditions.

### Example 16

E. coli BW25113sucA::FRT and BW25113sucA::FRTaceA::FRT having increased homocitrate production were constructed as follows. E. coli BW25113sucA::FRT-kan-FRT (JWO715-2) and BW25113aceA::FRT-kan-FRT (JW3875-3) were obtained from CGSC collection. Primers KL071 (SEQ ID NO: 39) and KL072 (SEQ ID NO: 40) were used to amplify the kanamycin resistant gene region flanking with homology regions from BW25113aceA::FRT-kan-FRT. This amplified DNA was electroporated into BW25113sucA::FRT/pKD46 to generate BW25113sucA::FRTaceA::FRT-kan-FRT. The kan genes in BW25113sucA::FRT-kan-FRT and BW25113sucA::FRTaceA::FRT-kan-FRT were removed from the chromosome using the FLP recombinase (pCP20). All the steps during the knockout process were monitored by PCR using primers KL069/070 (SEQ ID NOs: 41/42) and KL073/074 (SEQ ID NOs: 43/44).

It was confirmed that a sucA mutant E. coli lacking alpha-ketoglutarate dehydrogenase activity required succinate for aerobic growth on glucose minimal medium. In addition, it was demonstrated that BL21sucA::FRT had slower growth in complex medium supplemented with glucose compared to wild-type BL21. Supplementation of succinate at 10 mM concentration restored growth of this mutant in both minimal and complex medium. Furthermore, the sucAaceA double mutation completely abolished growth even in complex medium. Again, succinate supplementation at 10 mM in the medium restored growth of this mutant in both minimal and complex medium.

### Example 17

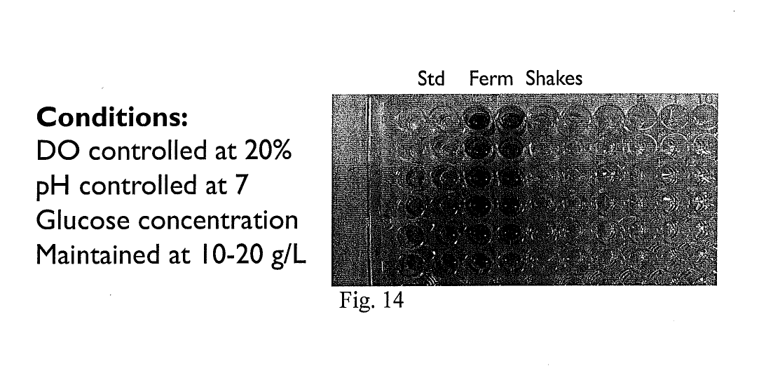

The carbon flux towards alpha-ketoglutarate production was examined using E. coli BW25113 and BW25113sucA::FRT under shake flasks conditions, which is aerobic but provides limited oxygen supply to the culture. A commercially available alpha-ketoglutarate bioassay kit (US Biological) was used to detect alpha-ketoglutarate in the medium. No significant amount of alpha-ketoglutarate was detected, as shown in FIG. 14 by low color intensity in the wells labeled “Shakes.”

The same strains were evaluated again in defined fermentation medium using Sartorius B-DCU fermentation system with 2 L working volume. Dissolved oxygen was maintained at 20% saturation by altering agitation (100-1000 rpm) as well as oxygen supplementation to the air stream (0-100%, air flow=333 ccm). The pH was controlled at 7.0 by the automatic addition of KOH (5 N). Glucose (60%) solution was added to the tank to maintain a final concentration between 10-20 g/L. As shown in FIG. 14, higher color intensity was observed for fermentation samples, thus indicating higher alpha-ketoglutarate concentration in the supernatant. By comparing to a standard calibration curve, alpha-ketoglutarate concentration in the fermentation supernatant was estimated to be 0.1 g/L.

All patents, published patent applications, publications and the subject matter mentioned therein are incorporated herein by reference. The publications discussed herein are provided solely for their disclosure prior to the filing date of this disclosure. Nothing herein is to be construed as an admission that this application is not entitled to antedate such publication by virtue of prior invention.

Although our processes have been described in connection with specific steps and forms thereof, it will be appreciated that a wide variety of equivalents may be substituted for the specified elements and steps described herein without departing from the spirit and scope of this disclosure as described in the appended claims.

1. A method of increasing production of a difunctional alkane in a recombinant host cell that produces a difunctional alkane from an alpha-ketoacid precursor comprising:
a) providing a difunctional alkane-producing recombinant host cell wherein the host cell has a deficiency in alpha-ketoglutarate dehydrogenase activity;
b) producing the difunctional alkane in the host cell.

2. The method of claim 1, wherein the recombinant cell exhibits an increase in activity of isocitrate lyase compared to a parent cell.

3. The method of claim 1, wherein the recombinant host cell underexpresses alpha-ketoglutarate dehydrogenase.

4. The method of claim 1, wherein the recombinant host cell does not express alpha-ketoglutarate dehydrogenase.

5. The method of claim 1, wherein the alpha-ketoglutarate dehydrogenase has a sequence having 80% identity with SEQ ID NO: 48.

6. The method of claim 2, wherein the isocitrate lyase has a sequence having 80% identity with SEQ ID NO: 49.

7. The method of claim 1, wherein the recombinant host cell further has a deficiency in activity of a regulatory protein encoded by arcA.

8. The method of claim 1, wherein the recombinant host cell further has a deficiency in activity of one or more enzymes selected from the group consisting of pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (ldhA).

9. The method of claim 1, wherein the recombinant host cell further has a deficiency in activity of at least two or more enzymes selected from the group consisting of pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (ldhA).

10. The method of claim 1, wherein the alpha-ketoacid is alpha-ketoglutarate.

11. The method of claim 1, wherein the difunctional alkane is a difunctional hexane.

12. The method of claim 1, wherein the difunctional alkane is adipic acid.

13. The method of claim 1, wherein the recombinant host cell further expresses at least one protein selected from the group consisting of citrate synthase with reduced sensitivity to NADH and pyruvate dehydrogenase with reduced sensitivity to NADH.

14. The method of claim 1, wherein the recombinant host cell further overexpresses acetyl-CoA synthetase.

15. A method of increasing production of a difunctional alkane in a recombinant host cell that produces a difunctional alkane from an alpha-ketoacid comprising:
a) providing a difunctional alkane-producing recombinant host cell wherein the host cell expresses at least one protein selected from the group consisting of (a) citrate synthase with reduced sensitivity to NADH and (b) pyruvate dehydrogenase with reduced sensitivity to NADH; and
b) producing the difunctional alkane in the host cell.

16. The method of claim 15, wherein the citrate synthase with reduced sensitivity to NADH has a sequence having 80% identity with SEQ ID NO: 51 or SEQ ID NO: 52.

17. The method of claim 15, wherein the citrate synthase with reduced sensitivity to NADH is a citrate synthase comprising an R163L amino acid mutation.

18. The method of claim 15, wherein the pyruvate dehydrogenase with reduced sensitivity to NADH has a sequence having 80% identity with SEQ ID NO: 54.

19. The method of claim 15, wherein the pyruvate dehydrogenase with reduced sensitivity to NADH is a pyruvate dehydrogenase comprising an E354K amino acid mutation.

20. A method of increasing production of a difunctional alkane in a recombinant host cell that produces a difunctional alkane from an alpha-ketoacid comprising:
a) providing a difunctional alkane-producing recombinant host cell wherein the host cell overexpresses acetyl-CoA synthetase; and
b) producing the difunctional alkane in the host cell.

21. A recombinant host cell for the increased production of a difunctional alkane from an alpha-ketoacid, wherein the host cell is a difunctional alkane-producing cell and has a deficiency in alpha-ketoglutarate dehydrogenase activity.

22. The host cell of claim 21, wherein the cell exhibits an increase in activity of isocitrate lyase compared to a parent cell.

23. The host cell of claim 21, wherein the cell overexpresses acetyl-CoA synthetase.

24. The host cell of claim 21, further comprising at least one protein selected from the group consisting of citrate synthase with reduced sensitivity to NADH and pyruvate dehydrogenase with reduced sensitivity to NADH.

25. The host cell of claim 21, wherein the cell has a deficiency in activity of at least one protein selected from the group consisting of isocitrate lyase (aceA), alpha-ketoglutarate dehydrogenase (sucA), the regulatory protein arcA, pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (ldhA).

26. The host cell of claim 21, wherein the alpha-ketoglutarate dehydrogenase has a sequence having 80% identity with SEQ ID NO: 48.

27. The host cell of claim 22, wherein the isocitrate lyase has a sequence having 80% identity with SEQ ID NO: 49.

28. The host cell of claim 21, wherein the engineered cell further has a deficiency in activity of a regulatory protein encoded by arcA.

29. The host cell of claim 21, wherein the engineered cell further has a deficiency in the activity of one or more enzymes selected from the group consisting of pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (ldhA).

30. The host cell of claim 21, wherein the engineered cell further has a deficiency in the activity of at least two or more enzymes selected from the group consisting of pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (ldhA).

31. The host cell of claim 21, wherein the alpha-ketoacid is alpha-ketoglutarate.

32. The host cell of claim 21, wherein the difunctional alkane is a difunctional hexane.

33. The host cell of claim 21, wherein the difunctional alkane is adipic acid.


SEQUENCE LISTING (55 sequences)

SEQ ID NO: 1 - protein, 384 aa, Azotobacter vinelandii
MASVIIDDTTLRDGEQSAGVAFNADEKIAIARALAELGVPELEIGIPSMGEEEREVMHAIAGLGLSSRLLAWCRLCDVDLAAARSTGVTMVDLSLPVSDLMLHHKLNRDRDWALREVARLVGEARMAGLEVCLGCEDASRADLEFVVQVGEVAQAAGARRLRFADTVGVMEPFGMLDRFRFLSRRLDMELEVHAHDDFGLATANTLAAVMGGATHINTTVNGLGERAGNAALEECVLALKNLHGIDTGIDTRGIPAISALVERASGRQVAWQKSVVGAGVFTHEAGIHVDGLLKHRRNYEGLNPDELGRSHSLVLGKHSGAHMVRNTYRDLGIELADWQSQALLGRIRAFSTRTKRSPQPAELQDFYRQLCEQGNPELAAGGMA

SEQ ID NO: 2 - DNA, 1155 bp, Azotobacter vinelandii
atggcgtcagtcattatcgatgacaccacgctgcgtgatggcgaacagtcggctggtgtggcgtttaacgccgatgaaaaaattgctatcgcgcgtgcgctggcagaactgggtgttccggaactggaaattggcatcccgagtatgggtgaagaagaacgtgaagtcatgcatgctattgcgggcctgggtctgagctctcgtctgctggcgtggtgccgcctgtgtgatgtggacctggcggcggcacgctccaccggtgtgacgatggttgatctgtcactgccggtttcggacctgatgctgcatcacaaactgaatcgtgatcgtgactgggcactgcgtgaagttgcacgcctggtcggcgaagcacgtatggctggtctggaagtgtgcctgggctgtgaagatgcgtctcgcgccgacctggaatttgtggttcaggtcggtgaagtggcacaggctgcaggtgctcgtcgcctgcgttttgcggataccgttggtgtcatggaaccgttcggcatgctggatcgttttcgcttcctgagccgtcgcctggacatggaactggaagtgcatgcgcacgatgacttcggtctggcaaccgcaaacacgctggcagcagtgatgggtggtgcaacccatattaacaccacggttaatggcctgggtgaacgtgcaggcaacgctgcgctggaagaatgcgttctggctctgaaaaatctgcacggcattgataccggtatcgacacgcgcggtattccggcaatcagcgctctggtggaacgtgcatctggccgccaggttgcctggcaaaaaagtgtcgtgggcgcgggtgtcttcacccatgaagccggcatccacgtggatggtctgctgaaacatcgtcgcaactatgaaggtctgaatccggatgaactgggccgcagtcactccctggttctgggcaaacatagcggtgcacacatggtccgtaacacgtaccgcgatctgggtattgaactggcagactggcagtctcaagctctgctgggccgtatccgcgcctttagtacccgtacgaaacgttccccgcagccggcagaactgcaagatttctatcgccagctgtgtgaacaaggtaatccggaactggccgcaggcggtatggcctaa

SEQ ID NO: 3 - protein, 338 aa, Methanosarcina barkerii
MRLAVIEGDGIGREIIPAAVKVLDAFGLEFEKVPLELGYTRWERTGTAISNNDLETIKGCDAVLFGAITTVPDPNYKSVLLTIRKELDLYANVRPVKPLPGITGVTGRNDFDFIIVRENTEGLYSGIEEIGPELSWTKRVVTRKGSERIAEYACKLAKKRKNKLTIVHKSNVLKSDKLFLDVCRQTASANGVEYEDMLVDSMAYNLIMRPERYDIVVTTNLFGDILSDMCAALVGSLGLVPSANIGEKYAFFEPVHGSAPDIAGKGIANPLAAILCVKMLLEWMGEPRSQIIDEAIAYVLQKKIITPDLGGTASTMEVGNAVAEYVLSNIQDRRSPPW

SEQ ID NO: 4 - protein, 339 aa, Methanosarcina maripaludis
MRNTPKICVINGDGIGNEVIPETVRVLNEIGDFEFIETHAGYECFKRCGDAIPEKTIEIAKESDSILFGSVTTPKPTELKNKPYRSPILTLRKELDLYANIRPTFNFKNLDFVIIRENTEGLYVKKEYYDEKNEVATAERIISKFGSSRIVKFAFDYALQNNRKKVSCIHKANVLRITDGLFLGVFEEISKKYEKLGIVSDDYLIDATAMYLIRNPQMFDVMVTTNLFGDILSDEAAGLIGGLGMSPSANIGDKNGLFEPVHGSAPDIAGKGISNPIATILSAAMMLDHLKINKEAEYIRNAVKKTVECKYLTPDLGGHLKTSEVTEKIIESIKSQMIQ

SEQ ID NO: 5 - DNA, 1017 bp, Methanosarcina barkerii
atgcgtctggcggttattgaaggcgatggtatcggccgcgaaattatcccggcggccgttaaagtcctggacgcctttggcctggaatttgaaaaagtgccgctggaactgggctatacccgttgggaacgcaccggtacggcaattagcaacaatgatctggaaacgatcaaaggctgcgacgcggtcctgtttggtgccattaccaccgtgccggacccgaattataaaagcgtgctgctgaccatccgtaaagaactggacctgtacgctaacgtgcgcccggttaaaccgctgccgggtattaccggcgtcacgggtcgtaacgattttgacttcattatcgttcgcgaaaataccgaaggcctgtatagcggtattgaagaaatcggcccggaactgtcttggaccaaacgtgtggttacgcgcaaaggtagcgaacgtattgcggaatacgcctgcaaactggcgaaaaaacgtaaaaacaaactgaccatcgtccataaaagcaatgtgctgaaatctgataaactgtttctggacgtgtgtcgtcagacggcaagtgctaacggcgtggaatatgaagatatgctggttgacagcatggcgtataatctgattatgcgtccggaacgctacgatatcgtcgtgaccacgaacctgttcggtgatattctgtcagacatgtgcgcagctctggttggcagtctgggtctggtcccgtccgcaaatatcggcgaaaaatacgcgtttttcgaaccggtgcacggttccgcaccggatattgctggtaaaggcatcgcgaacccgctggcggccattctgtgtgttaaaatgctgctggaatggatgggcgaaccgcgctcacagattatcgatgaagcgatcgcctatgtgctgcagaagaaaattatcaccccggatctgggcggcaccgcctcgacgatggaagtcggtaacgcagtggctgaatacgttctgtcaaatattcaagatcgtcgctcgccgccgtggtaa

SEQ ID NO: 6 - DNA, 1020 bp, Methanosarcina maripaludis
atgcgtaataccccgaaaatctgtgttatcaacggcgacggtatcggcaatgaagttatcccggaaacggtgcgtgtgctgaatgaaattggcgattttgaatttatcgaaacccatgcgggctatgaatgctttaaacgttgtggtgatgcaattccggaaaaaacgattgaaatcgctaaagaaagtgactccatcctgttcggttcagtcaccacgccgaaaccgaccgaactgaaaaacaaaccgtatcgttcgccgattctgacgctgcgcaaagaactggatctgtacgccaatatccgtccgaccttcaacttcaaaaacctggacttcgtgatcatccgcgaaaacacggaaggcctgtacgttaaaaaagaatactacgatgagaaaaacgaagtcgcgaccgccgaacgtattatcagcaaattcggtagctctcgcattgtgaaatttgcgttcgattatgcgctgcaaaacaaccgtaaaaaagtttcttgcatccacaaagcgaacgtcctgcgcatcaccgacggcctgtttctgggtgtgttcgaagaaattagtaaaaaatacgaaaaactgggcattgtttccgatgactatctgatcgatgcaacggctatgtacctgatccgtaacccgcaaatgtttgacgtgatggttaccacgaacctgtttggtgatattctgtcagacgaagcggcgggtctgatcggcggtctgggcatgtcaccgtcggccaacattggcgataaaaatggtctgtttgaaccggttcatggctccgcaccggacattgctggcaaaggtatcagcaatccgattgcaaccatcctgagcgcggcgatgatgctggatcacctgaaaattaacaaagaagcggaatatatccgcaatgccgtgaagaaaaccgtggaatgtaaatacctgacgccggatctgggcggtcatctgaaaaccagcgaagttaccgaaaaaattatcgaaagtattaaaagccaaatgattcagtga

SEQ ID NO: 7 - protein, 418 aa, Methanococcus maripaludis
MTLAEKIISKNVGKNVYAGDSVEIDVDVAMTHDGTTPLTVKAFEQISDKVWDNEKIVIIFDHNIPANTSKAANMQVITREFIKKQGIKNYYLDGEGICHQVLPEKGHVKPNMIIAGADSHTCTHGAFGAFATGFGATDMGYVYATGKTWLRVPETIQVNVTGENENISGKDIILKTCKEVGRRGATYLSLEYGGNAVQNLDMDERMVLSNMAIEMGGKAGIIEADDTTYKYLENAGVSREEILNLKKNKIKVNESEENYYKTFEFDITDMEEQIACPHHPDNVKGVSEVSGIELDQVFIGSCTNGRLNDLRIAAKHLKGKKVNESTRLIVIPASKSIFKEALKEGLIDTFVDSGALICTPGCGPCLGAHQGVLGDGEVCLATTNRNFKGRMGNTKSEVYLSSPAIAAKSAVKGYITNE

SEQ ID NO: 8 - protein, 161 aa, Methanococcus maripaludis
MKITGKVHVFGDDIDTDAIIPGAYLKTTDEYELASHCMAGIDEDFPEMVKEGDFLVAGENFGCGSSREQAPIAIKYCGIKAIIVESFARIFYRNCINLGVFPIECKGISKHVKDGDLIELDLENKKVILKDKVLDCHIPTGTAKDIMDEGGLINYAKKQKN

SEQ ID NO: 9 - DNA, 1257 bp, Methanococcus maripaludis
atgacgctggcagaaaaaattatctccaaaaatgtgggtaaaaatgtctatgcgggtgactcggttgaaattgatgttgatgtggcaatgacccatgacggcaccacgccgctgacggttaaagcctttgaacagattagtgataaagtttgggacaacgaaaaaatcgtcattatcttcgatcacaacattccggcgaatacctccaaagcggccaacatgcaggtcattacccgtgaatttatcaaaaaacaaggtatcaaaaactattacctggatggcgaaggtatctgccatcaagtgctgccggaaaaaggccacgttaaaccgaacatgattatcgcgggtgccgactcacatacctgtacgcacggcgcctttggtgcattcgctaccggcttcggtgcaacggatatgggctatgtttacgctaccggtaaaacgtggctgcgcgtcccggaaaccattcaagtgaatgttacgggcgaaaacgaaaacatctccggtaaagatattatcctgaaaacctgcaaagaagttggccgtcgcggtgcaacgtatctgagcctggaatacggcggtaacgctgtgcaaaatctggatatggacgaacgtatggttctgtctaacatggctattgaaatgggcggtaaagccggcattatcgaagccgatgacaccacgtataaatacctggaaaatgcaggtgtctcacgcgaagaaatcctgaacctgaagaaaaacaaaatcaaagtgaacgaatcggaagaaaactactacaaaaccttcgaatttgatattacggacatggaagaacagattgcgtgcccgcatcacccggataacgtcaaaggcgtgagtgaagtttccggtatcgaactggaccaagtgtttattggctcatgtaccaacggtcgtctgaatgatctgcgcattgcagctaaacatctgaaaggcaaaaaagtcaatgaatcgacccgtctgattgtgatcccggcgagcaaatctatctttaaagaagccctgaaagaaggcctgattgataccttcgttgacagcggtgcgctgatctgcacgccgggctgcggtccgtgtctgggcgcccaccagggtgtcctgggtgatggtgaagtgtgtctggcaaccacgaaccgtaatttcaaaggccgcatgggtaataccaaatctgaagtgtatctgtcgtcaccggcaatcgcagctaaatcagcagtcaaaggttacatcaccaatgaataa

SEQ ID NO: 10 - DNA, 486 bp, Methanococcus maripaludis
atgaaaattacgggcaaagtccacgtcttcggcgatgacattgacacggatgcgattattccgggtgcttatctgaaaaccacggatgaatatgaactggcaagccattgcatggctggtatcgatgaagactttccggaaatggtcaaagaaggcgattttctggtggcgggtgaaaacttcggctgcggtagctctcgtgaacaggcgccgattgccatcaaatattgtggcattaaagcgattatcgtggaaagttttgcccgtattttctaccgcaactgcatcaatctgggcgttttcccgattgaatgtaaaggtatctccaagcatgtgaaagatggcgacctgattgaactggatctggaaaacaaaaaagtgatcctgaaagataaagttctggactgtcacattccgaccggtacggcgaaagacattatggatgaaggtggcctgattaactacgctaaaaaacagaaaaactaa

SEQ ID NO: 11 - protein, 420 aa, Methanocaldococcus jannaschii
MTLVEKILSKKVGYEVCAGDSIEVEVDLAMTHDGTTPLAYKALKEMSDSVWNPDKIVVAFDHNVPPNTVKAAEMQKLALEFVKRFGIKNFHKGGEGICHQILAENYVLPNMFVAGGDSHTCTHGAFGAFATGFGATDMAYIYATGETWIKVPKTIRVDIVGKNENVSAKDIVLRVCKEIGRRGATYMAIEYGGEVVKNMDMDGRLTLCNMAIEMGGKTGVIEADEITYDYLKKERGLSDEDIAKLKKERITVNRDEANYYKEIEIDITDMEEQVAVPHHPDNVKPISDVEGTEINQVFIGSCTNGRLSDLREAAKYLKGREVHKDVKLIVIPASKKVFLQALKEGIIDIFVKAGAMICTPGCGPCLGAHQGVLAEGEICLSTTNRNFKGRMGHINSYIYLASPKIAAISAVKGYITNKLD

SEQ ID NO: 12 - protein, 170 aa, Methanocaldococcus jannaschii
MIIKGRAHKFGDDVDTDAIIPGPYLRTTDPYELASHCMAGIDENFPKKVKEGDVIVAGENFGCGSSREQAVIAIKYCGIKAVIAKSFARIFYRNAINVGLIPIIANTDEIKDGDIVEIDLDKEEIVITNKNKTIKCETPKGLEREILAAGGLVNYLKKRKLIQSKKGVKT

SEQ ID NO: 13 - DNA, 1263 bp, Methanocaldococcus jannaschii
atgaccctggtggaaaaaatcctgagcaaaaaagtgggctatgaagtttgcgctggtgattctattgaagttgaagtcgatctggcgatgacgcatgacggcaccacgccgctggcatacaaagctctgaaagaaatgagcgattctgtttggaacccggacaaaatcgtggttgcatttgatcacaacgtcccgccgaataccgtgaaagcggccgaaatgcagaaactggccctggaatttgtgaaacgtttcggcatcaaaaacttccataaaggcggtgaaggtatctgccaccaaattctggccgaaaactatgttctgccgaatatgtttgtcgcaggcggtgatagccatacctgtacgcacggcgcttttggtgcattcgctaccggcttcggtgcgacggacatggcctatatctacgcaaccggcgaaacgtggattaaagttccgaaaaccatccgtgtcgatattgtgggtaaaaacgaaaatgtctctgcgaaagacatcgttctgcgcgtctgcaaagaaattggccgtcgcggtgctacctatatggcgatcgaatacggcggtgaagtcgtgaaaaacatggatatggacggccgtctgacgctgtgtaatatggccattgaaatgggcggtaaaaccggtgttatcgaagcagatgaaattacgtatgactacctgaaaaaagaacgcggtctgtcagatgaagacatcgcgaaactgaaaaaagaacgtattaccgtgaaccgcgatgaagccaactactacaaagaaatcgaaatcgatatcacggacatggaagaacaggtggcggttccgcatcacccggataacgtgaaaccgatttcagacgttgaaggcaccgaaatcaaccaagtgtttattggctcatgtacgaatggtcgtctgtcggatctgcgcgaagcagctaaatatctgaaaggtcgcgaagtccataaagacgtgaaactgatcgttattccggcttcgaaaaaagtctttctgcaggcgctgaaagaaggcattatcgatatcttcgtgaaagcgggtgccatgatttgcaccccgggttgcggtccgtgtctgggtgcacatcaaggtgttctggcagaaggcgaaatttgtctgagtaccacgaaccgtaatttcaaaggccgcatgggtcacatcaacagttatatttacctggcctccccgaaaatcgcggccatttccgcagtgaaaggttacattaccaataaactggattaa

SEQ ID NO: 14 - DNA, 513 bp, Methanocaldococcus jannaschii
atgattattaaaggccgtgcccacaaatttggcgacgatgttgacaccgatgcgattattccgggtccgtatctgcgtaccaccgacccgtatgaactggcaagtcattgcatggctggcatcgatgaaaacttcccgaaaaaagtgaaagaaggcgacgtgatcgttgcaggtgaaaatttcggctgcggtagctctcgtgaacaggcagttatcgctatcaaatactgtggtatcaaagcggtcatcgccaaatcttttgcgcgtattttctaccgcaacgccattaatgttggcctgatcccgattatcgcgaacacggatgaaattaaagatggtgacattgtcgaaatcgatctggacaaagaagaaatcgtgatcaccaacaaaaacaaaacgatcaaatgtgaaaccccgaaaggcctggaacgtgaaatcctggcagccggcggtctggtcaactatctgaaaaaacgcaaactgatccaaagcaaaaaaggtgtgaaaacgtaa

SEQ ID NO: 15 - DNA, 128 bp, Artificial Sequence
  Description of Artificial Sequence Synthetic polynucleotide
aaatcatgaaaaatttatttgctttgtgagcggataacaattataatagcatgctggtcagtattgagcgatgcatgcacggtttccctctagaaataattttgtttaacttttaggaggtaaaaatc

SEQ ID NO: 16 - protein, 548 aa, Lactococcus lactis
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 17 - DNA, 1647 bp, Lactococcus lactis
atgtataccgtgggcgactacctgctggaccgtctgcatgaactgggcatcgaagaaatctttggtgtgccgggcgactataacctgcaatttctggatcagattatctcgcataaagacatgaaatgggttggtaacgcaaatgaactgaacgcatcttatatggctgatggctacgcgcgtaccaaaaaagcggcggcgtttctgaccacgttcggcgttggtgaactgagtgcggtcaacggcctggccggttcctatgcagaaaatctgccggtggttgaaattgtgggcagcccgacgtctaaagttcagaatgagggtaaatttgtccatcacaccctggcggatggcgactttaaacatttcatgaaaatgcacgaaccggtcacggctgcgcgtaccctgctgacggcggaaaacgcaaccgtcgaaattgatcgtgtgctgagtgccctgctgaaagaacgcaaaccggtgtacatcaatctgccggttgacgtcgccgcagctaaagcagaaaaaccgagcctgccgctgaaaaaagaaaacagtacctccaatacgtccgatcaggaaattctgaacaaaatccaagaatcactgaaaaatgccaaaaaaccgattgttatcacgggccacgaaattatctcctttggtctggaaaaaaccgtcacgcagttcatttcaaaaaccaaactgccgatcaccacgctgaactttggtaaaagctctgttgatgaagcgctgccgagcttcctgggcatttataacggtaccctgtctgaaccgaatctgaaagaatttgtggaaagtgctgatttcatcctgatgctgggcgttaaactgaccgacagttccacgggtgcgtttacccatcacctgaacgaaaataaaatgattagcctgaacatcgatgaaggtaaaatcttcaacgaacgtatccagaacttcgatttcgaatcactgatttcatcgctgctggacctgtcggaaatcgaatacaaaggcaaatacatcgataaaaaacaagaagacttcgtgccgagtaatgccctgctgtcccaggatcgcctgtggcaagcagtcgaaaacctgacgcagtcgaatgaaaccattgtggctgaacaaggcaccagctttttcggtgcgagctctatctttctgaaatcaaaatcgcatttcattggtcagccgctgtggggctccatcggttatacctttccggcggcactgggcagccaaattgctgataaagaatctcgtcacctgctgttcatcggcgacggttctctgcaactgacggtgcaagaactgggtctggccattcgtgaaaaaatcaacccgatctgctttatcatcaacaatgatggctacaccgttgaacgcgaaattcatggtccgaaccagagctataatgacatcccgatgtggaattactcaaaactgccggaatcgtttggcgccacggaagatcgtgtcgtgtctaaaattgtgcgcaccgaaaacgaatttgtgagtgttatgaaagaagcacaggctgacccgaatcgcatgtattggattgaactgatcctggcaaaagaaggcgcaccgaaagtcctgaaaaaaatgggtaaactgttcgctgaacaaaataaatcgtaa

SEQ ID NO: 18 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 19 - DNA, 1644 bp, Lactococcus lactis
atgtacaccgtgggcgactatctgctggaccgtctgcatgaactgggcattgaagaaatctttggcgttccgggcgactataacctgcagtttctggatcaaattatctcacgtgaagacatgaaatggattggtaacgcaaatgaactgaacgcatcgtatatggctgatggctacgcgcgcaccaaaaaagcggccgcatttctgaccacgttcggcgttggtgaactgagcgcgattaacggcctggccggttcttatgcagaaaatctgccggtggttgaaatcgttggctcaccgacgtcgaaagtccagaatgatggtaaatttgtgcatcacaccctggcggatggcgacttcaaacattttatgaaaatgcacgaaccggtgacggctgcgcgtaccctgctgacggcggaaaacgccacctatgaaattgatcgtgtgctgagtcagctgctgaaagaacgcaaaccggtttacatcaatctgccggttgacgtcgccgcagctaaagctgaaaaaccggcgctgtccctggaaaaagaaagctctaccacgaacaccacggaacaggttattctgagcaaaatcgaagaatctctgaaaaatgcccaaaaaccggtcgtgattgcaggccatgaagtgatcagttttggtctggaaaaaaccgtcacgcagttcgtgtccgaaaccaaactgccgattaccacgctgaactttggtaaaagcgccgtggatgaaagcctgccgtctttcctgggcatttataacggtaaactgagtgaaatctccctgaaaaacttcgtcgaatctgctgatttcatcctgatgctgggcgtgaaactgaccgacagttccacgggtgcctttacccatcacctggatgaaaacaaaatgattagcctgaatatcgacgaaggcatcatcttcaacaaagttgtcgaagatttcgacttccgtgcggtggtttcatcgctgtctgaactgaaaggcattgaatatgaaggccagtacatcgataaacaatacgaagaatttatcccgagcagcgcaccgctgagtcaggaccgtctgtggcaagcagttgaatcactgacgcagtcgaacgaaaccattgtcgctgaacaaggcaccagctttttcggtgcgtccaccatctttctgaaaagtaattcccgtttcattggtcagccgctgtggggcagcatcggttatacctttccggcggcactgggctcacaaattgccgataaagaatcgcgccatctgctgttcatcggcgacggcagcctgcaactgaccgttcaagaactgggtctgtcgattcgtgaaaaactgaacccgatctgctttattatcaacaatgatggctacacggtggaacgcgaaattcacggtccgacccagagttataacgacatcccgatgtggaattactccaaactgccggaaacgtttggcgcaaccgaagatcgtgtcgtgagcaaaattgtgcgcaccgaaaacgaatttgtgtctgttatgaaagaagcacaggctgatgttaatcgcatgtattggatcgaactggtcctggaaaaagaagatgctccgaaactgctgaaaaaaatgggcaaactgttcgctgaacaaaataaataa

SEQ ID NO: 20 - protein, 477 aa, Acinetobacter sp.
MNYPNIPLYINGEFLDHTNRDVKEVFNPVNHECIGLMACASQADLDYALESSQQAFLRWKKTSPITRSEILRTFAKLAREKAAEIGRNITLDQGKPLKEAIAEVTVCAEHAEWHAEECRRIYGRVIPPRNPNVQQLVVREPLGVCLAFSPWNFPFNQAIRKISAAIAAGCTIIVKGSGDTPSAVYAIAQLFHEAGLPNGVLNVIWGDSNFISDYMIKSPIIQKISFTGSTPVGKKLASQASLYMKPCTMELGGHAPVIVCDDADIDAAVEHLVGYKFRNAGQVCVSPTRFYVQEGIYKEFSEKVVLRAKQIKVGCGLDASSDMGPLAQARRMHAMQQIVEDAVHKGSKLLLGGNKISDKGNFFEPTVLGDLCNDTQFMNDEPFGPIIGLIPFDTIDHVLEEANRLPFGLASYAFTTSSKNAHQISYGLEAGMVSINHMGLALAETPFGGIKDSGFGSEGGIETFDGYLRTKFITQLN

SEQ ID NO: 21 - DNA, 1434 bp, Acinetobacter sp.
atgaactatccgaacatcccgctgtacatcaacggcgaatttctggaccataccaatcgtgacgtgaaggaagtctttaacccggtgaaccatgaatgcattggcctgatggcgtgtgccagtcaggcggatctggactacgctctggaaagctctcagcaagcctttctgcgttggaaaaagaccagtccgattacccgtagcgaaatcctgcgcaccttcgcaaaactggctcgtgaaaaggcggccgaaattggccgcaatatcaccctggatcagggtaaaccgctgaaggaagcaattgctgaagttacggtctgcgcggaacatgccgaatggcacgcagaagaatgtcgtcgcatttatggccgtgttatcccgccgcgcaacccgaatgtccagcaactggtggttcgtgaaccgctgggtgtgtgcctggcattttcaccgtggaactttccgttcaatcaggctattcgcaaaatctcggcagctattgcggcgggttgtaccattatcgtgaaaggctcaggtgatacgccgtcggcggtttatgcgattgcccaactgttccacgaagccggcctgccgaacggtgttctgaatgtcatctggggtgatagtaactttatttccgactacatgatcaaaagcccgattatccagaaaattagctttaccggctcgacgccggttggtaaaaagctggcaagccaggcgagcctgtatatgaaaccgtgcacgatggaactgggcggccatgcaccggtgattgtttgtgatgacgccgatattgacgcagctgtggaacacctggttggctacaaatttcgtaatgccggtcaggtctgcgtgtcaccgacgcgcttctacgtccaagaaggtatctacaaggaattttctgaaaaggtcgtgctgcgtgcaaaacagatcaaggttggctgtggtctggatgcgagttccgacatgggcccgctggcacaagctcgtcgcatgcatgcaatgcagcaaattgtcgaagatgctgtgcacaaaggtagtaagctgctgctgggcggtaacaaaatctccgacaagggcaactttttcgaaccgaccgtgctgggtgatctgtgcaacgacacgcagtttatgaatgatgaaccgttcggcccgattatcggtctgattccgtttgataccatcgaccatgttctggaagaagccaaccgtctgccgtttggtctggcaagctatgccttcaccacgtcatcgaaaaacgcgcatcagattagctacggcctggaagccggtatggtgtctatcaatcacatgggtctggcgctggccgaaaccccgtttggcggtattaaagatagcggcttcggttctgaaggcggcattgaaaccttcgacggctacctgcgtaccaaatttattacccaactgaactga

SEQ ID NO: 22 - DNA, 33 bp, Artificial Sequence
  Description of Artificial Sequence Synthetic primer
cacccgggagaaggagatatacatatgaccctg

SEQ ID NO: 23 - DNA, 29 bp, Artificial Sequence
  Description of Artificial Sequence Synthetic primer
gcatcgattatgcggccgtgtacaatacg

SEQ ID NO: 24 - DNA, 33 bp, Artificial Sequence
  Description of Artificial Sequence Synthetic primer
ccggatcctaccatggcgtcagtcattatcgat

SEQ ID NO: 25 - DNA, 40 bp, Artificial Sequence
  Description of Artificial Sequence Synthetic primer
ctagaagcttcctaaagcaggttaggccataccgcctgcg

SEQ ID NO: 26 - DNA, 37 bp, Artificial Sequence
  Description of Artificial Sequence Synthetic primer
gcgtataatatttgcccattgtgaaaacgggggcgaa

SEQ ID NO: 27 - DNA, 37 bp, Artificial Sequence
  Description of Artificial Sequence Synthetic primer
gtctttcattgccatacgaaattccggatgagcattc

SEQ ID NO: 28 - DNA, 41 bp, Artificial Sequence
  Description of Artificial Sequence Synthetic primer
cgaccccgggaagcttcgatgataagctgtcaaacatgaga

SEQ ID NO: 29 - DNA, 41 bp, Artificial Sequence
  Description of Artificial Sequence Synthetic primer
cgatggatccgatatctcacttattcaggcgtagcaccagg

SEQ ID NO: 30 - DNA, 36 bp, Artificial Sequence
  Description of Artificial Sequence Synthetic primer
cgaggatcctcatgattattaaaggccgtgcccaca

SEQ ID NO: 31 - DNA, 41 bp, Artificial Sequence
  Description of Artificial Sequence Synthetic primer
tctagatatcaagctttctagaaacgaaaggcccagtcttt

SEQ ID NO: 32 - DNA, 41 bp, Artificial Sequence
  Description of Artificial Sequence Synthetic primer
atccgatatcggatccgagctccatgcacagtgaaatcata

SEQ ID NO: 33 - DNA, 42 bp, Artificial Sequence
  Description of Artificial Sequence Synthetic primer
gccgcggatccctcgagttaatccagtttattggtaatatag

SEQ ID NO: 34 - DNA, 35 bp, Artificial Sequence
  Description of Artificial Sequence Synthetic primer
atatccttaagctcgagcagctggcggccgcttat

SEQ ID NO: 35 - DNA, 37 bp, Artificial Sequence
  Description of Artificial Sequence Synthetic primer
cgctgaattcacatgtataccgtgggcgactacctgc

SEQ ID NO: 36 - DNA, 42 bp, Artificial Sequence
  Description of Artificial Sequence Synthetic primer
cgtgcggccgcctcgagttacgatttattttgttcagcgaac

SEQ ID NO: 37 - DNA, 40 bp, Artificial Sequence
  Description of Artificial Sequence Synthetic primer
cgttcaggaattggatcctataccgtgggcgactacctgc

SEQ ID NO: 38 - DNA, 40 bp, Artificial Sequence
  Description of Artificial Sequence Synthetic primer
cgttcaggaattggatcctacaccgtgggcgactatctgc

SEQ ID NO: 39 - DNA, 35 bp, Artificial Sequence
  Description of Artificial Sequence Synthetic primer
gaagaacgtttctcccaggggcgttttgacgatgc

SEQ ID NO: 40 - DNA, 35 bp, Artificial Sequence
  Description of Artificial Sequence Synthetic primer
cctacgttcggcaacggctgtaggcatgataagac

SEQ ID NO: 41 - DNA, 35 bp, Artificial Sequence
  Description of Artificial Sequence Synthetic primer
gcgcgccatcggccatatcaagtcgatgttgttgc

SEQ ID NO: 42 - DNA, 35 bp, Artificial Sequence
  Description of Artificial Sequence Synthetic primer
ctttaatggcgctggcgtcgagattgtgttcagcc

SEQ ID NO: 43 - DNA, 35 bp, Artificial Sequence
  Description of Artificial Sequence Synthetic primer
gagcaatggcaaaccggtgaccaaagccttgttcc

SEQ ID NO: 44 - DNA, 35 bp, Artificial Sequence
  Description of Artificial Sequence Synthetic primer
cggcatttagcgccctcatcaggagcagagaattg

SEQ ID NO: 45 - protein, 556 aa, Escherichia coli
MSVSAFNRRWAAVILEALTRHGVRHICIAPGSRSTPLTLAAAENSAFIHHTHFDERGLGHLALGLAKVSKQPVAVIVTSGTAVANLYPALIEAGLTGEKLILLTADRPPELIDCGANQAIRQPGMFASHPTHSISLPRPTQDIPARWLVSTIDHALGTLHAGGVHINCPFAEPLYGEMDDTGLSWQQRLGDWWQDDKPWLREAPRLESEKQRDWFFWRQKRGVVVAGRMSAEEGKKVALWAQTLGWPLIGDVLSQTGQPLPCADLWLGNAKATSELQQAQIVVQLGSSLTGKRLLQWQASCEPEEYWIVDDIEGRLDPAHHRGRRLIANIADWLELHPAEKRQPWCVEIPRLAEQAMQAVIARRDAFGEAQLAHRICDYLPEQGQLFVGNSLVVRLIDALSQLPAGYPVYSNRGASGIDGLLSTAAGVQRASGKPTLAIVGDLSALYDLNALALLRQVSAPLVLIVVNNNGGQIFSLLPTPQSERERFYLMPQNVHFEHAAAMFELKYHRPQNWQELETAFADAWRTPTTTVIEMVVNDTDGAQTLQQLLAQVSHL

SEQ ID NO: 46 - protein, 568 aa, Oxlobacter formigenes
MSNDDNVELTDGFHVLIDALKMNDIDTMYGVVGIPITNLARMWQDDGQRFYSFRHEQHAGYAASIAGYIEGKPGVCLTVSAPGFLNGVTSLAHATTNCFPMILLSGSSEREIVDLQQGDYEEMDQMNVARPHCKASFRINSIKDIPIGIARAVRTAVSGRPGGVYVDLPAKLFGQTISVEEANKLLFKPIDPAPAQIPAEDAIARAADLIKNAKRPVIMLGKGAAYAQCDDEIRALVEETGIPFLPMGMAKGLLPDNHPQSAAATRAFALAQCDVCVLIGARLNWLMQHGKGKTWGDELKKYVQIDIQANEMDSNQPIAAPVVGDIKSAVSLLRKALKGAPKADAEWTGALKAKVDGNKAKLAGKMTAETPSGMMNYSNSLGVVRDFMLANPDISLVNEGANALDNTRMIVDMLKPRKRLDSGTWGVMGIGMGYCVAAAAVTGKPVIAVEGDSAFGFSGMELETICRYNLPVTVIIMNNGGIYKGNEADPQPGVISCTRLTRGRYDMMMEAFGGKGYVANTPAELKAALEEAVASGKPCLINAMIDPDAGVESGRIKSLNVVSKVGKK

SEQ ID NO: 47 - protein, 528 aa, Pseudomonas putida [sequence omitted]

SEQ ID NO: 48 - protein, 933 aa, Escherichia coli
MQNSALKAWLDSSYLSGANQSWIEQLYEDFLTDPDSVDANWRSTFQQLPGTGVKPDQFHSQTREYFRRLAKDASRYSSTISDPDTNVKQVKVLQLINAYRFRGHQHANLDPLGLWQQDKVADLDPSFHDLTEADFQETFNVGSFASGKETMKLGELLEALKQTYCGPIGAEYMHITSTEEKRWIQQRIESGRATFNSEEKKRFLSELTAAEGLERYLGAKFPGAKRFSLEGGDALIPMLKEMIRHAGNSGTREVVLGMAHRGRLNVLVNVLGKKPQDLFDEFAGKHKEHLGTGDVKYHMGFSSDFQTDGGLVHLALAFNPSHLEIVSPVVIGSVRARLDRLDEPSSNKVLPITIHGDAAVTGQGVVQETLNMSKARGYEVGGTVRIVINNQVGFTTSNPLDARSTPYCTDIGKMVQAPIFHVNADDPEAVAFVTRLALDFRNTFKRDVFIDLVCYRRHGHNEADEPSATQPLMYQKIKKHPTPRKIYADKLEQEKVATLEDATEMVNLYRDALDAGDCVVAEWRPMNMHSFTWSPYLNHEWDEEYPNKVEMKRLQELAKRISTVPEAVEMQSRVAKIYGDRQAMAAGEKLFDWGGAENLAYATLVDEGIPVRLSGEDSGRGTFFHRHAVIHNQSNGSTYTPLQHIHNGQGAFRVWDSVLSEEAVLAFEYGYATAEPRTLTIWEAQFGDFANGAQVVIDQFISSGEQKWGRMCGLVMLLPHGYEGQGPEHSSARLERYLQLCAEQNMQVCVPSTPAQVYHMLRRQALRGMRRPLVVMSPKSLLRHPLAVSSLEELANGTFLPAIGEIDELDPKGVKRVVMCSGKVYYDLLEQRRKNNQHDVAIVRIEQLYPFPHKAMQEVLQQFAHVKDFVWCQEEPLNQGAWYCSQHHFREVIPFGASLRYAGRPASASPAVGYMSVHQKQQQDLVNDALNVE

SEQ ID NO: 49 - protein, 434 aa, Escherichia coli
MKTRTQQIEELQKEWTQPRWEGITRPYSAEDVVKLRGSVNPECTLAQLGAAKMWRLLHGESKKGYINSLGALTGGQALQQAKAGIEAVYLSGWQVAADANLAASMYPDQSLYPANSVPAVVERINNTFRRADQIQWSAGIEPGDPRYVDYFLPIVADAEAGFGGVLNAFELMKAMIEAGAAAVHFEDQLASVKKCGHMGGKVLVPTQEAIQKLVAARLAADVTGVPTLLVARTDADAADLITSDCDPYDSEFITGERTSEGFFRTHAGIEQAISRGLAYAPYADLVWCETSTPDLELARRFAQAIHAKYPGKLLAYNCSPSFNWQKNLDDKTIASFQQQLSDMGYKFQFITLAGIHSMWFNMFDLANAYAQGEGMKHYVEKVQQPEFAAAKDGYTFVSHQQEVGTGYFDKVTTIIQGGTSSVTALTGSTEESQF

SEQ ID NO: 50 - protein, 238 aa, Escherichia coli
MQTPHILIVEDELVTRNTLKSIFEAEGYDVFEATDGAEMHQILSEYDINLVIMDINLPGKNGLLLARELREQANVALMFLTGRDNEVDKILGLEIGADDYITKPFNPRELTIRARNLLSRTMNLGTVSEERRSVESYKFNGWELDINSRSLIGPDGEQYKLPRSEFRAMLHFCENPGKIQSRAELLKKMTGRELKPHDRTVDVTIRRIRKHFESTPDTPEIIATIHGEGYRFCGDLED

SEQ ID NO: 51 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 52 - protein, 372 aa, Bacillus subtilis
MTATRGLEGVVATTSSVSSIIDDTLTYVGYDIDDLTENASFEEIIYLLWHLRLPNKKELEELKQQLAKEAAVPQEIIEHFKSYSLENVHPMAALRTAISLLGLLDSEADTMNPEANYRKAIRLQAKVPGLVAAFSRIRKGLEPVEPREDYGIAENFLYTLNGEEPSPIEVEAFNKALILHADHELNASTFTARVCVATLSDIYSGITAAIGALKGPLHGGANEGVMKMLTEIGEVENAEPYIRAKLEKKEKIMGFGHRVYKHGDPRAKHLKEMSKRLTNLTGESKWYEMSIRIEDIVTSEKKLPPNVDFYSASVYHSLGIDHDLFTPIFAVSRMSGWLAHILEQYDNNRLIRPRADYTGPDKQKFVPIEERA

SEQ ID NO: 53 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 54 - protein, 474 aa, Escherichia coli [sequence omitted]

SEQ ID NO: 55 - protein, 474 aa, Klebsiella pneumoniae
MSTEIKTQVVVLGAGPAGYSAAFRCADLGLETVIVERYSTLGGVCLNVGCIPSKALLHVAKVIEEAKALAEHGIVFGEPKTDIDKIRTWKEKVITQLTGGLAGMAKGRKVKVVNGLGKFTGANTLEVEGENGKTVINFDNAIIAAGSRPIQLPFIPHEDPRVWDSTDALELKSVPKRMLVMGGGIIGLEMGTVYHALGSEIDVVEMFDQVIPAADKDVVKVFTKRISKKFNLMLETKVTAVEAKEDGIYVSMEGKKAPAEAQRYDAVLVAIGRVPNGKNLDAGKAGVEVDDRGFIRVDKQMRTNVPHIFAIGDIVGQPMLAHKGVHEGHVAAEVISGLKHYFDPKVIPSIAYTEPEVAWVGLTEKEAKEKGISYETATFPWAASGRAIASDCADGMTKLIFDKETHRVIGGAIVGTNGGELLGEIGLAIEMGCDAEDIALTIHAHPTLHESVGLAAEVFEGSITDLPNAKAKKK